(19) United States
(12) Patent Application Publication
(10) Pub. No.: US 2018/0042627 A1
(43) Pub. Date: Feb. 15, 2018

(54) Adaptive Lithotripsy For Cancer Risk Reduction

(71) Applicants: Dennis W. Gilstad, San Antonio, TX (US); Barbara C. Gilstad, San Antonio, TX (US)

(72) Inventors: Dennis W. Gilstad, San Antonio, TX (US); Barbara C. Gilstad, San Antonio, TX (US)

(21) Appl. No.: 15/645,430

(22) Filed: Jul. 10, 2017

Related U.S. Application Data
(62) Division of application No. 15/235,131, filed on Aug. 12, 2016, now abandoned.

(51) Int. Cl. A61B 17/22

(52) U.S. Cl. CPC A61B 17/22029; A61B 2017/22005

(57) ABSTRACT

Adaptive lithotripsy systems assist diagnosis and treatment of patients with kidney stones (stones being associated with subsequent development of cancer). As stimulation vibration is transmitted to the patient, both its total transmitted power and power spectral density (PSD) are tailored to individual patient needs. One such need is for progressive stone fragmentation (a hallmark of adaptive lithotripsy systems) at minimum power levels. And minimum power levels are achieved through two adaptive mechanisms for shifting PSD to concentrate transmitted vibration power in more effective frequency ranges. This concentration necessarily reduces power in relatively ineffective ranges, thus minimizing collateral tissue damage. Effective ranges for vibration power concentration are estimated in near-real time using backscatter vibration that is retransmitted from resonating stones while encoding information on the stones' existence, size and composition. Backscatter vibration thus informs adaptive tailoring of stimulation vibration for lithotripsy that is (1) relatively safer and (2) more efficient.

The invention relates generally to medical treatments for reducing cancer risk. Such treatments often involve minimally-invasive applications of energy (e.g., thermal ablation, phototherapy) and/or diagnostic procedures involving removal of suspect tissue (e.g., skin biopsies). The invention is related to U.S. Pat. Nos. 8,939,200, 9,027,636, and 9,169,707 and co-pending U.S. patent application Ser. No. 14/918,848 (filed 22 Oct. 2015).

### FIELD OF THE INVENTION

The invention specifically relates to reducing the risk of cancer associated with kidney stones (herein, renal calculi). Note that the term renal calculi herein may also include ureter stones and fragments of stones. Many case reports have suggested an association of renal calculi with cancer. And a recent long-term study has indicated that individuals hospitalized for renal calculi are at increased risk of developing renal pelvis/ureter or bladder cancer, even beyond 10 years of follow-up. Suspicion has centered on the role of chronic irritation (and associated infections) that may lead to proliferative urothelial changes, including cancer. See, e.g., Chow et al., Risk of Urinary Tract Cancers Following Kidney or Ureter Stones. J Natl Cancer Inst, 1997; 89:1453-7.

### INTRODUCTION

Adaptive lithotripsy systems are described herein for minimizing chronic tissue irritation and associated infections due to renal calculi. The invention relies on innovative applications of well-known technical principles to achieve timely, efficient and minimally-invasive diagnosis and treatment for reducing cancer risk. And adaptive lithotripsy is characterized by superior patient safety features when compared with alternatives including: ureteroscopy, conventional extracorporeal shock wave lithotripsy (ESWL) or burst wave lithotripsy (BWL).

Adaptive lithotripsy treatments employ systems comprising one or more adaptive stimulators. An adaptive stimulator comprises a tunable vibration generator and at least one vibration sensor, the generator transmitting bursts of vibration power generated by mechanical shocks (i.e., shock-waves). Each shock-wave is associated with a kinetic energy impulse produced by an electromagnetically movable hammer striking, flexing, and rebounding from, a fluid interface during a reflex cycle time. Hence, impulse-generated vibration. Shock-waves are generally associated with broad spectrum transmitted vibration power (i.e., transmitted vibration spectra comprising a relatively large range of frequencies, typically comprising both sonic and ultrasonic frequencies). The duration of reflex cycle time is related to the frequency content of transmitted vibration spectra as follows: relatively faster rebounds, meaning shorter reflex cycle times, are associated with relatively broader transmitted vibration spectra (i.e., comprising relatively larger ranges of vibration frequencies).

Total transmitted vibration power from tunable vibration generators is thus in the form of bursts of vibration, each burst comprising a relatively large range (or spectrum) of vibration frequencies. And adaptive lithotripsy systems include provisions for near-real-time closed-loop feedback-control of (1) total transmitted vibration power and (2) the power spectral density (PSD) of transmitted vibration spectra. PSD reflects the distribution of vibration power within the spectrum of transmitted vibration frequencies.

Feedback-control electrical signals needed to implement feedback-control of transmitted vibration power are produced in programmable stimulator controllers running frag diagnostics to process vibration electrical signals from vibration detectors. Vibration detectors are responsive to both transmitted and characteristic backscatter vibration, the latter radiating from renal calculi as they absorb transmitted vibration power, vibrate at their resonant frequencies, fracture and subsequently fragment. And transmitted vibration is distinguished from characteristic backscatter vibration in vibration electrical signals via several parameters (e.g., amplitude, PSD, and/or timing relative to hammer strikes).

Note that when adaptive lithotripsy is applied to a specific patient and results in generation of (and detection of) characteristic backscatter vibration, the patient likely has renal calculi. So characteristic backscatter vibration (and/or feedback control electrical signals derived therefrom) can be used as diagnostic indicators in screening tests for patients who, though asymptomatic, may nevertheless have renal calculi (thus likely predisposing them to cancer).

In support of its diagnostic features, feedback-control in adaptive lithotripsy also facilitates maximally-efficient, minimal-power stimulation of vibration in renal calculi to generate characteristic backscatter vibration which reveals the presence of the calculi. In other words, transmitted vibration spectra containing resonant frequencies of renal calculi are applied to the patient at minimum effective power levels. Following a positive diagnosis based on characteristic backscatter vibration from the calculi, a treatment plan will be developed for adaptive lithotripsy at higher stimulation power which fosters efficient absorption of transmitted vibration power by the calculi, followed by their fracture and subsequent fragmentation (while limiting collateral tissue damage). Fragmented calculi, of course, are eliminated with the normal flow of urine.

In an adaptive lithotripsy system, feedback control of the PSD of transmitted vibration spectra facilitates continuous adjustment (i.e., up-shifting or down-shifting) of the transmitted vibration's frequency content. Such PSD shifting is accomplished in part by smooth (e.g., continuous) changes in the reflex cycle time associated with the tunable vibration generator's moving hammer as it strikes, flexes, and rebounds from, the fluid interface. PSD shifting is also a function of the (continuously magnetostrictively adjustable) resonant frequencies of the shock-wave generator's fluid interface.

So alteration of a shock-wave generator's reflex cycle time and/or fluid interface resonant frequencies can affect PSD up-shifting or down-shifting. This means feedback control can concentrate (or shift) transmitted vibration power within the relatively narrow resonant frequency spectra that are needed to efficiently fragment renal calculi at any given time. In other words, renal calculi are exposed to vibration power spectra that are continuously adjusted in near-real time to facilitate adaptive stimulation in a vibration frequency range approximating that which will most efficiently predispose individual calculi to absorb transmitted vibration power, vibrate at their resonant frequencies, radiate characteristic backscatter vibration as they fracture and subsequently fragment. See, e.g., U.S. Pat. No. 8,695,476, incorporated by reference.

Efficient fragmentation of calculi, in turn, allows for reductions in total transmitted vibration power and/or shortening the duration of a treatment. Reducing total transmitted vibration power is accomplished by reducing the hammer's velocity as it strikes the fluid interface of a tunable vibration generator.

Note that in adaptive lithotripsy, the effectively narrowed resonant vibration frequency spectra needed for efficient stimulation of renal calculi are typically transmitted hydraulically. And effectively narrowed spectra are generated in nearly real-time by concentration of (i.e., tailoring of) transmitted vibration power within relatively broad vibration spectra as they are impulse-generated. See, e.g., U.S. Pat. No. 4,756,192, incorporated by reference

Summarizing, impulse generation of relatively broad vibration spectra is described herein as resulting from a movable hammer striking a fluid interface, hammer movement being responsive to an electromagnetic hammer driver and/or to a longitudinal magnetic field generated by current in a peripheral coil surrounding the fluid interface and transverse to the longitudinal axis. The transmitted vibration frequency spectra are effectively narrowed, as described herein, by concentrating (or shifting) relative vibration power into the most efficient frequency ranges for fragmentation of renal calculi. Such effective narrowing of the frequency spectra shifts the PSD of transmitted vibration power by moving transmitted vibration power out of the remainder of the broad spectrum of impulse-generated vibration. Achieving effective narrowing (or tailoring) of transmitted vibration power in the present invention employs two mechanisms: (1) up-shifting or down-shifting PSD via continuous electromagnetic adjustment of the tunable vibration generator's reflex cycle time and/or (2) continuous magnetostrictive adjustment of the tunable vibration generator's fluid interface resonant (i.e., natural) frequencies. Further tailoring of transmitted vibration includes adjustment of total transmitted vibration power by altering a hammer's velocity as it strikes a fluid interface.

Note that shock waves produced by a movable hammer striking a fluid interface are more amenable (through adjustment of reflex cycle time) to the tailoring operations described herein than shock waves produced by other means (e.g., electric spark or electric heating of an electrolyte). See, e.g., U.S. Pat. Nos. 9,352,294 and 6,383,152, incorporated by reference.

PSD up-shifting increases relative transmitted vibration power in higher frequencies, while PSD down-shifting increases relative transmitted vibration power in lower frequencies. Such PSD shifts allow continuous (i.e., swept-frequency) tailoring of total impulse-generated resonant frequency power. This tailoring is accomplished in near-real time to maximize resonance vibration in the renal calculi being stimulated (thus maximizing their absorption of transmitted vibration power with fracture and subsequent fragmentation into smaller sizes).

Note that the relatively higher resonant frequencies of smaller size calculi tend to announce their own existence as a lithotripsy treatment progresses. The announcement is in the form of characteristic backscatter vibration which originates in the fracturing calculi and is then hydraulically retransmitted as characteristic backscatter vibration energy. This backscatter vibration energy is sensed in near-real time by one or more vibration detectors associated with each tunable vibration generator, the detector(s) thus producing vibration electrical signals representative of both transmitted vibration and backscatter vibration.

Characteristic backscatter vibration, in turn, informs adaptive control of transmitted power PSD via one or more programmable stimulator controllers producing feedback control electrical signals. For the desired progressive stimulation of calculi, adaptive control is reflected in up-shifting transmitted vibration PSD as needed to efficiently achieve continuing fracturing and fragmentation of renal calculi. This is one objective of adaptive extracorporeal shock wave lithotripsy or AESWL as described herein. A second objective is low-cost, low-risk and high-accuracy diagnosis of renal calculi. See, e.g., U.S. Pat. No. 8,535,250, incorporated by reference.

Note that the process of fracturing renal calculi typically results in the presence of a range of fragment sizes at any given time. For this reason, the relatively broad spectrum of impulse-generated vibration frequencies is tailored in adaptive stimulators as noted above (via feedback-control in near-real time). Tailoring facilitates adaptive control of the range of transmitted vibration frequencies to approximate the range of natural resonant frequencies of the calculi present at any given time. The result is that the total vibration energy hydraulically transmitted by a tunable vibration generator is adaptively and efficiently transmitted (in near-real time) in frequency ranges comprising the most efficacious frequencies: (1) for transmission of the minimum effective amount of total vibration power from the generator to the renal calculi and (2) for maximizing efficient absorption of the transmitted vibration power by renal calculi (thus predisposing them to fracture and subsequently fragment at each stage of treatment). See, e.g., U.S. Pat. No. 9,339,284, incorporated by reference.

Note further that AESWL, as described herein, differs materially from burst wave lithotripsy (BWL). An example embodiment of the latter technique includes transmission of high intensity focused ultrasound at a fixed frequency (e.g., 200 kHz), while a first alternative embodiment features switching among a limited number of vibration generators, each transmitting pulses of fixed frequency vibration (e.g., 170, 285 and 800 kHz). A second alternative embodiment employs switching from transmission of one fixed frequency to transmission of another fixed frequency in a single generator. Any of these or analogous techniques can approximately match the (changing) resonance frequencies of a limited range of calculi sizes, at least for a short time. But the unavoidable mismatches of calculi resonance frequency and transmitted vibration frequency mean that significant amounts of transmitted vibration power are not efficiently absorbed as calculi sizes decrease during treatment. Further, transmitted vibration power that is not absorbed must be dissipated in the surrounding tissue with the potential for collateral damage to the patient. The results of BWL thus contrast sharply and unfavorably with the present invention's smooth near-real-time swept-frequency tailoring of vibration power distribution according to the changing needs of each patient as treatment progresses. See, e.g., U.S. Patent Application Publication 2013/0245444.

So it is unfortunate but unsurprising that adverse aspects of both ESWL and BWL are currently influencing a transition to ureteroscopy, notwithstanding the relatively invasive nature and troubling complication rates of the latter. The rationale for this transition would be dramatically altered if embodiments of the present invention were employed to (1) increase lithotripter efficacy and (2) reduce associated medical complications through more efficient vibration-induced fracture and subsequent fragmentation of renal calculi. These benefits are achievable in the adaptive lithotripsy systems described herein through analysis of characteristic backscatter vibration from fracturing calculi to inform adaptive closed-loop feedback-controlled control of both the PSD (by two mechanisms) and the total transmitted power of impulse-generated vibration spectra.

Implementation of feedback-control as described herein is a key advantage of the invention. In particular, adaptive impulse-type shock wave generators are made to (hydraulically) transmit broad-spectrum vibration which is altered through feedback-control of both frequency content and amplitude, the latter generally being significantly different in transmitted vibration compared with that of characteristic backscatter vibration. Feedback-controlled vibration, in turn, is associated with the tailored mechanical shocks of the invention's tunable vibration generators. The resultant controlled range of transmitted vibration frequencies can efficiently lead to the excitation of renal calculi representing a range of natural vibration resonances, thus predisposing calculi of various sizes to absorb vibration power as they resonate, then fracture and subsequently fragment. A variety of designs shown and described herein explain how such resonance-tuned fragmentation-inducing vibration can be controlled to promote predictable and efficient clearing of renal calculi.

Specific examples are cited in the following paragraphs to illustrate how designs for lithotripter reliability and performance improvements have evolved from a better understanding of causes and effects of shock and vibration in tunable shock-wave generators. First, remarkably strong and repetitive energy impulses (associated with mechanical shocks of a hammer striking a fluid interface) originate in the generators. Second, both the bandwidth and amplitude of impulse-generated vibration produced by electromagnetically driven hammers can be feedback controlled through alteration of reflex cycle time and/or hammer velocity at hammer strike (the latter being related to reflex cycle time and also a function of current in a transverse peripheral coil). Third, both the bandwidth and amplitude of hydraulically-transmitted impulse-generated vibration are also functions of the resonant frequencies of a shock wave generator's fluid interface (which can be adaptively altered magnetostrictively via current in a transverse peripheral coil). And Fourth, without implementation of innovative designs for near-real-time adjustment of total transmitted vibration power and its PSD, conventional lithotripters are, by comparison, relatively inefficient. That is, conventional lithotripters hydraulically transmit excessive vibration (and thus excessive energy) outside of desired power and resonant-frequency ranges for the target calculi. Such excess energy is not readily absorbed by calculi, so it must be dissipated elsewhere (as, for example, in causing collateral damage to the surrounding tissue).

To minimize collateral tissue damage, relatively broad-spectrum vibration originating in adaptive lithotripsy's tunable vibration generators can be tailored generally as described herein. In particular, tailoring can be initiated by alternately up-shifting and down-shifting (i.e., cyclically shifting) the power spectral densities (PSD's) of the vibration frequency spectra in a predetermined manner. Cyclical PSD shifting produces vibration frequency sweeps originating in the predictably-varying PSD's. The frequency sweeps thus embedded in stimulation vibration can facilitate maximization of stimulation efficiency through analysis of characteristic backscatter vibration originating in stimulated (e.g., fracturing) calculi.

To optimize stimulation functions, the extent of calculi fracturing is periodically assessed during progressive fragmentation in near-real time. Assessment begins with detection of characteristic (i.e., band-limited) backscatter vibration corresponding to the frequency sweeps of stimulation vibration. Such backscatter vibration emanates from the stimulated calculi as they fracture, and backscatter assessment analysis proceeds in near-real time. In particular, vibration electrical signals from vibration detectors sensing the characteristic backscatter vibration are processed in programmable controllers to produce feedback-control electrical signals. Note that signal processing in programmable controllers is carried out using empirically-derived software algorithms (broadly termed herein: frag diagnostics).

Note further that use of swept-frequency impulse-generated stimulation vibration confers significant advantages in characterizing renal calculi. First, the broad spectrum of transmitted vibration power ensures that a broad range of calculi sizes will resonate (and hence tend to fracture and subsequently fragment) with each burst of stimulation vibration energy. Then the PSD's and amplitude's of characteristic backscatter vibration not only reflect the extent of desired fracturing of calculi, but also the sizes and compositions of the fragments subsequently formed. Second, due in part to the electro-mechanical mode of stimulation vibration generation described herein, the bandwidth, phase and amplitude of vibration frequency sweeps will vary slightly from burst-to-burst. Inherently then, the likelihood of missing critical calculi resonance vibration frequencies within successive frequency sweeps of stimulation vibration is thereby reduced. Third, the stimulation vibration described herein can be tailored: e.g., its total transmitted vibration power and/or output PSD are closed-loop feedback-controlled. Thus, frequency sweeps can be adjusted to electively and effectively concentrate transmitted vibration power in progressively higher frequency ranges. And Fourth, the near-real-time concentration of stimulation energy in frequency ranges likely to induce desired fracturing of calculi of various sizes results in higher efficiency.

That is, progressive stimulation-induced fracturing of calculi with subsequent fragmentation is achieved at minimal levels of total transmitted vibration power. Stimulation energy thus applied to a patient minimizes collateral tissue damage because the relative amount of stimulation energy transmitted in less productive frequency ranges is reduced.

The above-described advantages of tailored stimulation stem in part from the fact that characteristic backscatter vibration, processed via frag diagnostics to yield feedback control electrical signals, provides newly-developed calculi-related and fragment-related information that is otherwise unobtainable.

The newly-developed information is extractable from vibration electrical signals that are produced by vibration detectors from characteristic backscatter vibration. Programmable stimulator controllers process vibration electrical signals to form of feedback control electrical signals which allow tailoring of the process of closed-loop stimulation to the requirements of individual patients.

Such closed-loop feedback-control of stimulation vibration incorporates feedback of a portion of the controlled-system output (i.e., characteristic backscatter vibration from stimulated calculi) to the controlled-system input (i.e., the process point where tailored stimulation vibration is generated via mechanical shock). In other words, information represented in characteristic backscatter vibration is used to alter both the mechanical shocks themselves and the resulting transmitted vibration spectra. The result is finely-tuned stimulation vibration adapted in near-real-time for quick convergence on optimal stimulation vibration frequency end-points.

Closed-loop feedback control of mechanical shocks in a tunable vibration generator as described herein implies control of the kinetic energy impulses corresponding to a moving hammer (or mass) striking, flexing, and rebounding from, a fluid interface in a generator. At least one such generator resides within each adaptive stimulator. And at least a portion of each generator's initial kinetic energy for each hammer strike is converted to broad-spectrum impulse-generated vibration energy which is sensed by at least one vibration detector which is paired with a generator in each adaptive stimulator. So with each hammer strike and rebound, the vibration spectrum's PSD and transmitted vibration power can be detected and adjusted within the stimulator's shock wave generator under closed-loop (local) control in near-real time.

Note that total transmitted vibration power is a function of hammer velocity as the hammer strikes (impacts on) the shock-wave generator's fluid interface. The vibration-related characteristics of the fluid interface (e.g., effective elastic modulus, resonant frequencies and damping) affect the ratio of vibration power transmitted to that dissipated (e.g., as heat). Thus, hammer velocity at hammer strike is only one of several adjustable (optimization) parameters, affecting transmitted vibration PSD and total transmitted power. And different optimization strategies may apply, depending on particular diagnostic and treatment applications of an adaptive lithotripsy system.

Optimization of closed-loop PSD control for adaptive stimulators means that hydraulically transmitted vibration spectra from a tunable vibration generator are tuned at their source (e.g., by electromagnetically altering the hammer velocity at impact and/or by altering the reflex cycle time for each hammer strike and/or by magnetostrictively altering the natural resonance frequencies of the stimulator's fluid interface). Such tuning effectively shapes a transmitted vibration spectrum's PSD to concentrate stimulation vibration power in predetermined frequency ranges. The predetermined frequency ranges for any stage of stimulation are: (1) ranges that maximize transmission of vibration resonance excitation power to the target calculi and/or (2) ranges that facilitate characterization of the target calculi through analysis of characteristic backscatter vibration. As stimulation proceeds, each predetermined range of transmitted vibration frequencies necessarily changes (through the mechanisms noted above), thereby generating frequency sweeps as described herein.

The stimulated calculi themselves report their actual absorption of tailored stimulation vibration energy by retransmitting (in the form of characteristic backscatter vibration) the energy associated with their resonant-vibration-induced fracturing. Feedback control electrical signals are then derived (in programmable controllers) from vibration electrical signals produced by vibration detectors for the characteristic backscatter vibration. Calculated feedback control electrical signals (which are functions of the vibration electrical signals) are transmitted from the controllers to one or more adaptive stimulators to close the loop in closed-loop impulse-generated vibration control while optimizing stimulation of calculi in near-real time.

The following background materials support this introduction by discussing the vibration spectrum of an impulse in greater detail, highlighting its importance with examples of deleterious effects of mechanical shock and vibration in conventional applications. Building on the background, subsequent sections describe selected alternative designs for adaptive lithotripsy system components.

### BACKGROUND

Insight into vibration-related fractures in calcului has been gained through review of earlier shock and vibration studies, data from which are cited herein. For example, a recent treatise on the subject describes a mechanical shock in terms of its inherent properties in the time domain and in the frequency domain, and also in terms of its effects on structures when the shock acts as the excitation. (see p. 20.5 of Harris' Shock and Vibration Handbook, Sixth Edition, ed. Allan G. Piersol and Thomas L. Paez, McGraw Hill (2010), hereinafter Harris).

References to time and frequency domains appear frequently in descriptions of acquisition and analysis of shock and vibration data. And these domains are mathematically represented on opposite sides of equations generally termed Fourier transforms. Further, estimates of a shock's structural effects are frequently described in terms of two parameters: (1) the structure's undamped natural frequency and (2) the fraction of critical structural damping or, equivalently, the resonant gain Q (see Harris pp. 7.6, 14.9-14.10, 20.10). (See also, e.g., U.S. Pat. No. 7,859,733, incorporated by reference).

Digital representations of time and frequency domain data play important roles in computer-assisted shock and vibration studies. In addition, shock properties are also commonly represented graphically as time domain impulse plots (e.g., acceleration vs. time) and frequency domain vibration plots (e.g., spectrum amplitude vs. frequency). Such graphical presentations readily illustrate the shock effects of hammer-strike energy impulses in a conventional generator. Relatively high acceleration values and broad vibration spectra are prominent, because each generator impulse response primarily represents a violent conversion of a portion of kinetic energy (of the moving hammer) to other energy forms during the hammer strike.

Since energy cannot be destroyed, and since an adaptive stimulator can neither store nor convert (i.e., dissipate) more than a small fraction of the moving hammer impulse's kinetic energy, a portion of that energy is necessarily transmitted via the stimulator's fluid interface in the form of broad-spectrum vibration energy.

Note that the relationship of (frequency domain) vibration energy to (time domain) kinetic energy, is mathematically represented by a Fourier transform. Such transforms are well-known to those skilled in the art of shock and vibration mechanics. For others, a graphical representation (i.e., plots) rather than a mathematical representation (i.e., equations) may be preferable.

For example, in a time domain plot, the transmitted energy appears as a high-amplitude impulse of short duration. And a corresponding frequency domain plot of transmitted energy reveals a relatively broad-spectrum band of high-amplitude vibration. ***The breadth of the vibration spectrum is generally inversely proportional to the impulse duration (see, e.g., reflex cycle time in adaptive stimulators).***

Thus, as noted above, a portion of the generator hammer's kinetic energy is converted to relatively broad-spectrum vibration energy. The overall effect of the mechanical shocks approximates the result of repeatedly striking the generator fluid interface with a commercially-available impulse hammer, each hammer strike being followed by flexing of the fluid interface and a hammer rebound (during a reflex cycle time). Impulse hammers are easily configured to produce relatively broad-spectrum high-amplitude excitation (i.e., vibration) in an object struck by the hammer. (See, e.g., Introduction to Impulse Hammers at http://www.dytran.com/img/tech/a11.pdf, and Harris p. 20.10).

Summarizing then, relatively broad-spectrum high-amplitude vibration predictably results from a typical high-energy hammer impact impulse. Thus, impulse-generated (e.g., hammer-generated) vibration occurs in bursts having relatively broad spectra simultaneously containing many vibration frequencies, typically ranging from a few Hz to several thousand Hz (kHz).

In conventional shock wave generators, nearly all of the (relatively broad-spectrum) impact-generated vibration energy must be transmitted to the patient because vibration energy cannot be efficiently dissipated in the generators themselves. Based on extensive shock and vibration test data (see Harris), impact-generated vibration will tend to excite any tissue it strikes within a relatively broad range of resonances. (See, e.g., U.S. Pat. No. 5,979,242, incorporated by reference). If a natural vibration resonance frequency of the tissue (e.g., bone, organ, vessel) coincides with a frequency within the transmitted vibration spectrum, collateral vibration damage may be significant.

### SUMMARY OF THE INVENTION

Adaptive lithotripsy systems employ one or more adaptive stimulators to provide impulse-generated swept-frequency stimulation vibration with provision for closed-loop feedback-control of both total transmitted vibration power and transmitted vibration PSD. Each adaptive stimulator comprises a tunable (electromechanical shock wave) vibration generator and at least one vibration detector.

Control of total transmitted vibration power includes adjustment of the (variable) hammer impact velocity on the fluid interface of a tunable vibration generator. And PSD control includes adjustment of variables including, e.g., hammer impact velocity and/or reflex cycle time and/or continuous magnetostrictive alteration of the fluid interface resonant frequencies of an adaptive stimulator's tunable vibration generator. Hammer impact velocity and reflex cycle time are controlled electromagnetically in the tunable vibration generator of an adaptive stimulator. And electromagnetic influences arise from the electromagnetic hammer driver and/or current in a transverse peripheral coil, the coil surrounding (and peripheral to) the fluid interface, while also being transverse to the longitudinal axis. The coil current is associated with a longitudinal magnetic field substantially perpendicular to the fluid interface. Note that in addition to the influences of variables cited above, the variables may interact with each other. Hence control and/or optimization strategies in general are empirically developed and represented as software in programmable stimulation controllers.

Total transmitted vibration power and PSD are controlled in adaptive lithotripsy so as to excite effective levels of resonant vibration in target renal calculi, thereby predisposing them to absorb transmitted vibration power, fracture and subsequently fragment. Closed-loop feedback-control uses characteristic backscatter vibration from resonating calculi to optimize PSD and/or total transmitted vibration power for efficient fragmentation of calculi at minimum (total transmitted) vibration power levels. Adaptive stimulators may be arranged singly or in spatial arrays of multiple adaptive stimulators, timed signals from a programmable stimulator controller altering directional propagation of combined vibration wave fronts from an adaptive stimulator array. See, e.g., the '200 patent.

Total transmitted power and PSD signals are closed-loop feedback-controlled, meaning that they use feedback derived from characteristic backscatter vibration from resonating (and fracturing) calculi to optimize total transmitted vibration power and/or PSD for (progressive) fragmentation of calculi. Whether adaptive stimulators are arranged singly or in spatial arrays comprising multiple adaptive stimulators, each adaptive stimulator transmits bursts of vibration spectra, each burst comprising a plurality of vibration frequencies within a predetermined range. Timed signals from a programmable stimulator controller alter directional propagation of combined-vibration wave fronts from an array. And as fragmentation of calculi proceeds to smaller fragments having higher resonant frequencies, PSD's are up-shifted, increasing relative transmitted vibration power in relatively higher frequency ranges to optimize progressive fragmentation efficiency. See, e.g., the '250 and '636 patents.

Optimization strategies for certain embodiments of adaptive lithotripsy systems combine (1) swept-frequency vibration arising from electromagnetically altered (e.g., cyclically-varying) reflex cycle times associated with impulse-generated stimulation vibration and/or (2) swept-frequency vibration arising from altered (e.g., cyclically-varying) tunable vibration generator fluid interface resonant frequencies (frequency variations being due to magnetostrictive alteration of the effective elastic modulus of one or more components of the fluid interface) and/or (3) altered (e.g., cyclically-varying) total transmitted vibration power to provide adaptive stimulation (transmitted power alterations being functions of hammer velocity just prior to hammer impact on the fluid interface). Note that optimizing adaptive lithotripsy systems requires estimates for each adaptive stimulator's cyclically-varying reflex cycle time associated with hammer strike and rebound in its tunable vibration generator (which includes estimates of hammer velocity at hammer strike). Hammer velocity estimates may be made, e.g., by estimators using table look-ups based on laboratory-generated data from fluid interface vibration tests, thus comparing real-time data from fluid interface vibration detectors to corresponding tabular data. More accurate estimates may be based, e.g., on real-time laser ranging of hammer movement (i.e., velocity and/or position) relative to the electromagnetic hammer driver.

Further, estimates are required for each adaptive stimulator's cyclically-varying fluid interface resonant frequencies, e.g., those sensed by one or more of the fluid interface's vibration detectors. Estimators may also rely on the fact that the fluid interface resonant frequencies are known functions or empirically-derived functions of current in the transverse coil. However the estimates are determined, estimators for reflex cycle time and resonant frequencies are electively designed into each programmable stimulator controller, or designed as stand-alone equipment that communicates with the programmable stimulator controller.

The controller, in turn, ensures that swept-frequency stimulation vibration arises in part from cyclical up-shifts and down-shifts of PSD achieved by electromechanical adjustment of reflex cycle time associated with hammer strikes in a shock wave generator. And swept-frequency stimulation vibration also arises in part from cyclical up-shifts and down-shifts of PSD achieved by magnetostrictive adjustment of shock-wave generator fluid interface resonant frequencies. The latter adjustment is achieved by changing the flux density of one or more longitudinal magnetic fields applied to one or more magnetostrictive amorphous ferromagnetic alloy disc-shaped thin members comprising a generator fluid interface. See, e.g., U.S. Pat. No. 8,093,869, incorporated by reference.

Among other remarkable properties of the above magnetostrictive amorphous ferromagnetic alloy disc-shaped thin members (comprising, e.g., the amorphous ferromagnetic alloy Metglas 2605SC), the disc-shaped thin members can be configured to resonate at a predetermined frequency and/or to convert applied mechanical energy to vibration electrical signals (as in, e.g., a vibration detector). See, e.g., U.S. Patent Application Publication 2005/0242955.

Magnetostrictive materials can also be configured as a magnetostrictive lens operable in response to a coil-generated magnetic field (see, e.g., U.S. Pat. No. 5,458,120, incorporated by reference). Metglas 2605SC exhibits a change up to about 80% of effective Young's Modulus (i.e., effective elastic modulus) with magnetization to saturation in bulk. Young's Modulus is an indicator of stiffness, and changes in stiffness can thus be used to tune the resonance frequency of a shock wave generator fluid interface. See, e.g., U.S. Pat. No. 5,381,068, incorporated by reference, and the '284 patent.

Both reflex cycle times and fluid interface resonant frequencies are modified via closed-loop control using feedback derived from characteristic backscatter vibration from resonating calculi. As noted above, adaptive stimulation for lithotripsy can be produced by combining a single tunable vibration generator with a vibration detector to form an adaptive stimulator. And a plurality of such stimulators spaced apart in a spatial array can transmit directionally-propagated combined-vibration wave fronts. A linear array, as schematically illustrated herein (see FIG. 5), is one type of spatial array. See, e.g., U.S. patent application number 2005/0038361, incorporated by reference

Whether singly or in a spatial array, each adaptive stimulator is under closed-loop feedback-control. And each stimulator responds to timed stimulator signals (e.g., timed stimulator transmission signals and/or timed stimulator PSD shift signals). Each stimulator transmits (in response to a timed stimulator transmission signal) an impulse-generated vibration burst comprising a plurality of vibration frequencies. And each such vibration burst has an adjustable total transmitted power as well as a power spectral density (PSD) which may be up-shifted or down-shifted under closed-loop feedback-control (via one or more stimulator shift signals) to create a swept-frequency spectrum. Connected array stimulators may be controlled by a periodic signal group comprising one or more signals for each stimulator in the array. That is, timed stimulator transmission signals and/or stimulator shift signals may be sent as timed signal groups from a programmable controller, at least one signal (either a transmission signal or a shift signal or both) for each stimulator. Signals within a timed signal group may be either simultaneous or sequential. Sequential stimulator signals are separated from each other by discrete time intervals within a signal group.

Stimulator shift signals control each stimulator's adjustable PSD by tuning via that stimulator's adjustable reflex cycle time and/or its fluid interface natural (resonant) vibration frequencies. For example, adjustable PSD is up-shifted (i.e., increasing relative transmitted power in higher vibration frequencies and decreasing relative transmitted power in lower vibration frequencies) by reducing hammer impact reflex cycle time and/or increasing fluid interface resonant frequencies. Down-shifting, in contrast, decreases the relative transmitted power of higher vibration frequencies and increases the relative transmitted power of lower vibration frequencies. And down-shifting is achieved by increasing hammer impact reflex cycle time and/or decreasing fluid interface resonant frequencies (e.g., by magnetostrictively altering the effective elastic modulus of one or more components of the fluid interface). Such tuning of one or more stimulators in a spatial array thus tunes the stimulation array as a whole for resonance excitation and fracturing of renal calculi.

Note that changes in reflex cycle times also affect vibration interference among stimulators within an array, while changes in stimulator transmission signal times (e.g. either simultaneous or sequential) can affect directional propagation of combined-vibration wave fronts from a stimulator array. Directional propagation of wave fronts from a stimulator array may augment (or be augmented by) directional propagation control secondary to one or more magnetostrictive lenses (see, e.g., the '120 patent).

As adaptive fracturing of renal calculi proceeds to smaller fragments having relatively higher resonant frequencies, adjustable stimulator PSD's are up-shifted to increase relative power in higher frequencies of their transmitted vibration. The PSD up-shifts are a function of characteristic backscatter vibration that (1) originates in fracturing calculi, (2) is then sensed in one or more vibration detectors which (3) communicate with one or more stimulator controllers via vibration electrical signals, the controller(s) being programmed to (4) produce feedback control electrical signals reflecting transmitted and/or characteristic backscatter vibration. Feedback control electrical signals are applied to the tunable vibration generators of adaptive stimulators in near-real time, thus increasing power in relatively higher frequencies of their transmitted vibration spectra so as to optimize progressive (adaptive) stimulation of calculi to resonance and fracture.

Smoothly controlled PSD up-shifts and down-shifts materially affect the relatively broad transmitted vibration frequency spectra of impulse-generated shock waves. Such relatively broad frequency spectra are transmitted by adaptive lithotripsy systems of the present invention, each spectrum comprising a plurality of frequencies. Such stimulation systems feature shock wave generators that electively combine three operational modes subject to control signals: (1) cyclically-varying fluid interface resonant frequencies and thus PSD, and/or (2) cyclically-varying hammer impact reflex cycle times and thus PSD, and/or (3) cyclically varying total transmitted vibration power. These three operational modes are synergistic for enabling optimization of overall adaptive lithotripsy efficiency.

Note that in addition to PSD up-shifts and down-shifts, stimulation vibration transmitted by a spatial array of adaptive stimulators may be subject to directional control. For example, a timed group of transmission control signals (e.g., simultaneous or sequential) directed to individual stimulators in a spatial array can result in transmission of a combined shock-wave front that is directionally governed by the timing of the control signals and the physical spacing of the individual stimulators. Directional shock wave control is thus achieved in a manner analogous to the operation of a phased-array antenna. Directionally controlled wave fronts, in turn, facilitate repeated scanning and characterization (via analysis of characteristic backscatter vibration) of renal calculi within effective range of a stimulator array.

Repeated characterizations of calculi at intervals, in turn, allow for adaptive stimulation that is continuously tailored (e.g., altered as to total transmitted vibration power and/or cyclical PSD shifts) to optimize fracturing of renal calculi in near-real time. In addition to the above alterations, such tailoring may comprise adjustment of phase relations among (1) stimulator shift signals (related to cyclical PSD shifts and the associated swept-frequency vibration) and/or (2) timed stimulator array transmission signals (related to directional control of combined-vibration bursts from generators of the stimulator array). The result is a parameter-rich control options environment for adaptive stimulation as described herein.

Control options as described herein facilitate stimulus vibration tailoring that is beneficially applied both early and repeatedly in the lithotripsy process. Since initial fracturing of renal calculi is generally associated with relatively large fragment sizes, early stimulus vibration tailoring emphasizes the relatively low resonant frequencies of these large fragments. Then the initial PSD down-shifting of transmitted stimulation vibration energy is typically followed by PSD up-shifting to encourage progressive fragmentation. These adaptations are, of course, feedback controlled in near-real time to optimize both the size and the timing of PSD shifts in light of actual fragmentation progress.

Such adaptations of transmitted stimulation vibration, however, also reflect a simultaneous need for minimizing adverse effects of transmitted vibration energy. Adverse effects include, e.g., energy loss and/or collateral damage in stimulated biologic tissue (i.e., stimulated entities) near the target renal calculi. To minimize such adverse effects, stimulation vibration is preferably applied as close to the calculi as practical, both to minimize transmission losses and also to minimize collateral tissue interactions and their potential for harm to the patient.

Collateral tissue interactions, of course, represent extraction of transmitted vibration energy which excites resonant vibrations in biologic features whose natural resonant frequencies were not precisely known initially. Since the extracted energy may lead to tissue damage, the damage is limited in use of the present invention by more specific characteristic-backscatter-vibration-informed tailoring to reduce the relative power in transmitted vibration of frequencies associated with collateral tissue resonances.

Specific tailoring is thus a function of characteristic backscatter vibration originating from any stimulated biologic entities. That is, specific tailoring depends on feedback derived from backscatter vibration which, on analysis, reflects the stimulation status, the position (including, e.g., distance from the generator) and/or the composition of the stimulated entities. Characteristic backscatter vibration is therefore sensed in near-real time by the vibration detectors of one or more adaptive stimulators. And analysis of vibration electrical signals from vibration detectors responsive to characteristic backscatter vibration, with subsequent feedback-controlled tailoring of transmitted vibration via feedback control electrical signals, ensures that stimulation vibration energy remains efficiently concentrated to achieve predetermined therapeutic goals while minimizing collateral damage to the patient.

Note that the detailed compositions of stimulated entities (such as renal calculi), and their reactions to stimulation vibration as reflected in characteristic backscatter vibration, typically demonstrate wide variations. After study and analysis via one or more programmable stimulator controllers, these variations provide supplemental data bearing on estimates of biologic composition that may be extracted in the controllers running empirically-derived frag diagnostics software.

Subsequent paragraphs consider generation of broad-spectrum vibration as it is adapted to therapeutic objectives in light of information derived from characteristic backscatter vibration. Consideration of both adaptive lithotripsy stimulators and systems comprising them emphasizes the role of feedback-mediated control in (1) generation of adaptive impulse-generated vibration for induced-resonance-detection, fracture and subsequent fragmentation of renal calculi and (2) maximizing the efficacy and safety of the relevant medical procedures.

Adaptive stimulators and their programmable controllers appear in adaptive systems schematically illustrated in FIGS. 4 and 5. Each figure represents system embodiments for generation and closed-loop feedback-control of adaptive stimulation. The illustrated system embodiments each comprise one or more adaptive stimulators, and each stimulator comprises a tunable (impulse) vibration generator and one or more vibration detectors. Adaptive stimulators create and transmit impulse-generated swept-frequency stimulation vibration, while receiving both transmitted vibration and characteristic backscatter vibration.

Note that each class 699 adaptive stimulator comprises one or more vibration detectors in the form of disc-shaped thin members as schematically shown in the exploded view of an adaptive stimulator (see FIG. 3). A different vibration detector embodiment (i.e., an accelerometer) is schematically illustrated in the exploded view of a class 599 adaptive stimulator embodiment in FIG. 2. In the adaptive stimulator embodiments of either FIG. 2 or FIG. 3, at least one vibration detector is built into the stimulator so as to detect (1) characteristic backscatter vibration from stimulated entities (e.g., renal calculi) and (2) impulse-generated vibration intended for transmission from the stimulator.

Each vibration detector in the closed-loop feedback-control system embodiments of FIGS. 4 and 5 is connected to a programmable stimulator controller which creates and transmits feedback control electrical signals as functions of characteristic backscatter and/or transmitted vibration as represented in vibration electrical signals from vibration detectors. Feedback control electrical signals, in turn, adapt each adaptive stimulator's transmitted vibration to changing operational requirements (e.g., changing transmitted power PSD and/or amplitude to support continuing progressive fracturing and fragmenting of renal calculi).

Additionally, the closed-loop feedback control systems of FIGS. 4 and 5 each comprises estimators for (1) each adaptive stimulator's cyclically-varying reflex cycle time associated with hammer strike and rebound in its tunable vibration generator (which includes estimates of hammer velocity at hammer strike) and (2) each adaptive stimulator's cyclically-varying fluid interface resonant frequencies. While both estimators are schematically illustrated as communicating with, but separate from, the (programmable) stimulator controller, either or both estimators may be incorporated in programmable controllers.

An adaptive stimulator of class 699 comprises least one disc-shaped thin member which functions as a vibration detector while its resonant frequencies are magnetostrictively responsive to applied magnetic fields. Three such disc-shaped thin members are schematically illustrated in the exploded view of FIG. 3, together forming a layered (or laminated) fluid interface of the class 699 adaptive stimulator. In addition to the disc-shaped thin members sensing vibration and/or altering the resonant frequencies of the fluid interface, they may also directly or indirectly affect a variety of fluid interface characteristics related to, e.g., structural integrity and/or damping.

Several fluid interface characteristics may be optimized through choices among thin member specialization parameters (e.g., varying thickness, composition, concavity and/or convexity). Damping optimization, for example, depends in part on parameters such as the Q (or quality) factor attributable to each fluid interface resonance. Q factors may be represented graphically on plots of amplitude vs. frequency. Such plots typically exhibit a single maximum at the local fluid interface resonance frequency, with decreasing amplitude values at frequencies above and below the resonance frequency. At amplitude values about 0.707 times the maximum value (i.e., the half-power point) the amplitude vs. frequency plot corresponds not to a single frequency but to a bandwidth between upper and lower frequency values on either side of the local fluid interface resonance. The quality factor Q is then estimated as the ratio of the resonance frequency to the bandwidth. See, e.g., pp. 2-18, 2-19 of Harris. See also U.S. Pat. No. 7,113,876, incorporated by reference.

Lower Q connotes the presence of more damping and a wider bandwidth (i.e., a relatively broader band of near-resonant frequencies on the amplitude vs. frequency plot). And higher Q connotes less damping and a narrower bandwidth, with the ideal case being zero damping and a single resonant frequency. Since ideal fluid interface resonances are not encountered in practice, optimization strategies for adaptive stimulators of class 699 typically include choice of the peak resonant frequency and Q of one or more thin members (in light of the desired peak resonance frequencies and Q's of the fluid interface of which they are a part). Resonant frequencies (e.g., those of vibration detectors or thin members) which are identified herein as “similar” to other resonant frequencies (e.g., fluid interface resonant frequencies) are thus understood to lie generally in the frequency range indicated by the upper and lower frequency values of the relevant Q response half-power bandwidth.

Fluid interface resonance frequencies are influenced by the presence and amount of damping, but are also controllable in-part via the magnetostrictive responsiveness of at least one disc-shaped thin member comprising one or more amorphous ferromagnetic alloys having desirable magnetostrictive properties (e.g., Metglas 2605SC). To achieve fluid interface resonant frequency control, all such disc-shaped thin (magnetostrictive) members forming the interface are peripherally enclosed by a coil form which itself encloses a transverse electromagnetic coil. The coil, when energized, produces a longitudinal magnetic field operative on the hammer (to alter reflex cycle time), and on each of the magnetostrictive disc-shaped thin members of the fluid interface (to alter their effective elastic modulus and thus alter their resonant frequencies).

At least one (and potentially all) disc-shaped thin (and magnetostrictively-responsive) members of the fluid interface (see, e.g., FIG. 3) communicate via vibration electrical signals with the adaptive stimulator's programmable controller. And the programmable controller is responsive to each such vibration electrical signal. That is, the programmable controller creates and transmits at least one feedback control electrical signal to the electromagnetic hammer driver and/or the transverse electromagnetic coil, as a function of each vibration electrical signal received from at least one of the disc-shaped thin members. Thus, the adaptive stimulator schematically illustrated in FIG. 3 functions to transmit relatively broad-spectrum, impulse-generated (via electromagnetic-control), and feedback-controlled vibration via a fluid interface having (magnetostrictively-responsive) adjustable resonant frequencies.

In greater detail and with reference to FIG. 3, each adaptive stimulator comprises a hammer longitudinally movable within a hollow cylindrical housing having a longitudinal axis, a first end, and a second end. The first end is closed by a fluid interface, and the fluid interface is surrounded by a coil form which encloses a transverse coil (i.e., a field emission structure) which is thus peripheral to the fluid interface. Current in the (transverse) peripheral coil creates a longitudinal magnetic field operative on the hammer (to alter reflex cycle time), and on each of the magnetostrictive disc-shaped thin members of the fluid interface (to alter their effective elastic modulus and thus alter their resonant frequencies).

Note that the longitudinal magnetic field is oriented generally along the longitudinal axis, although certain portions of the field are necessarily curved. Relatively small deviations from parallelism with the longitudinal axis of the fluid interface would be expected in any such field generated by a (transverse) peripheral coil.

Alterations of the fluid interface effective elastic modulus are facilitated by the fluid interface structure, i.e., a structure comprising one or more disc-shaped thin members (i.e., layers), each (relatively plane) disc shape being oriented substantially perpendicular to the longitudinal axis as schematically illustrated in FIG. 3. Note that the modifier “substantially perpendicular” for the orientation of each (relatively plane) disc shape is appropriate since portions of each disc surface may deviate somewhat from a precisely plane shape (e.g., due to slight convexity or concavity). Thus, relatively small deviations from perpendicularity to the longitudinal axis of an adaptive stimulator are expected in each such disc-shaped thin member's surface.

Nevertheless, the above noted alterations of the fluid interface effective elastic modulus arise from the composition of at least one disc-shaped thin member, i.e., comprising one or more amorphous ferromagnetic alloys. The effective elastic modulus of structures comprising such alloys is magnetostrictively responsive to the longitudinal magnetic field. Hence, at least one thin member comprises one or more amorphous ferromagnetic alloys and is configured as a vibration detector having an adjustable resonant frequency.

Further, the adaptive stimulator's cylindrical housing second end is closed by an electromagnetic hammer driver for an internal (longitudinally) sliding hammer. The electromagnetic hammer driver comprises at least one (non-laser) field emission structure (plus, optionally, a laser) for moving (and tracking the position and velocity of) the hammer. The hammer repeatedly strikes, and rebounds from, the fluid interface (during a reflex cycle time), generating a burst of broad-spectrum vibration (which is transmitted via the fluid interface) each time it does so.

Each of the above field emission structures is responsive to at least one feedback control electrical signal, meaning that one or more field parameters (e.g., coil current, electric field, magnetic field polarity and/or magnetic field strength) changes as a result of corresponding changes in the feedback control electrical signal(s). Further, the broad-spectrum vibration generated by an adaptive stimulator has a controllable PSD responsive to at least one feedback control electrical signal, meaning that the PSD shifts as a function of corresponding changes in the signal(s). In particular, each adaptive stimulator has adjustable (e.g., cyclically-varying) hammer impact reflex cycle time, hammer velocity at hammer strike, and/or fluid interface resonant frequencies, the latter altered by magnetostrictively changing the effective elastic modulus of one or more components of the fluid interface.

Hammer impact reflex cycle time and hammer velocity at hammer strike are influenced by the electromagnetic hammer driver and/or the longitudinal magnetic field, which may comprise, e.g., one or more electromagnetic field emission structures and/or one or more electric field emission structures. Since a hammer (i.e., a mass) is longitudinally movable within the cylindrical housing between the electromagnetic hammer driver and the fluid interface, such movement may be controlled in an open-loop or closed-loop manner. Control forces are exerted on the hammer via the magnetic and/or electrical fields of the field emission structure(s). (See, e.g., U.S. Pat. No. 8,760,252, incorporated by reference).

To facilitate hammer movement, the hammer may comprise, e.g., one or more permanent magnets, and the electromagnetic hammer driver's field emission structure(s) may comprise, e.g., one or more electromagnets, at least one with reversible polarity and variable field strength. See the '252 patent for other examples of field emission structures.

By design, the hammer periodically moves toward impact on the fluid interface, followed by flexing of the interface and movement of the hammer away from the fluid interface (i.e., rebounding from the impact). More specifically, the hammer moves (under the influence of the electromagnetic hammer driver's electric and/or magnetic fields and/or the longitudinal magnetic field) to strike, and rebound from, the fluid interface, thus generating broad-spectrum vibration. In other words, the cylindrical housing, electromagnetic hammer driver, transverse peripheral coil, hammer, and fluid interface can function together as a tunable vibration generator. By locating one or more vibration detectors within (or attached to) the fluid interface of the tunable vibration generator, an adaptive stimulator may be formed. The vibration detector(s) may comprise, e.g., (1) one or more disc-shaped thin members in the fluid interface, each of which is magnetostrictively responsive to vibration, or (2) one or more MEMS accelerometers mounted on the fluid interface. See, e.g., MicroElectro-Mechanical Systems in Harris, pp. 10-26, 10-27.

Note that the hammer is responsive to the electromagnetic hammer driver and/or the longitudinal magnetic field for striking, flexing, and rebounding from, the fluid interface. That is, the hammer may be, e.g., subject to magnetic attraction during certain portions of its longitudinal travel, and subject to magnetic repulsion during other portions of its longitudinal travel. Responsiveness of the hammer may be achieved via open-loop control (using empirically-derived predictions of hammer direction and velocity based, e.g., on field emission strength) or closed-loop control (using, e.g., feedback data on changes in hammer position to calculate direction and velocity of hammer movement). The latter data may be obtained, e.g., via laser ranging from the electromagnetic hammer driver and/or an electric field sensor on the fluid interface interacting with an electret electric field emission structure on the hammer.

In the class 699 adaptive stimulator of FIG. 3, electrical leads from each disc-shaped thin member and each field emission structure (e.g., the peripheral coil and the electromagnetic hammer driver) are combined in an electrical cable connected to a programmable stimulator controller. Taken together, the schematic illustrations and the written descriptions herein explain how a relatively less-complex conventional shock wave generator (which might be used in an open-loop lithotripsy system) is transformed into the functionally more-complex adaptive stimulator of the present invention to meet the greater demands of an adaptive closed-loop feedback-control lithotripsy system for reducing cancer risk.

Regardless of an adaptive stimulator's class (e.g., class 599 in FIGS. 1 and 2 or class 699 in FIG. 3), stimulation vibration energy is transmitted in relatively short bursts associated with hammer impacts on a fluid interface. Vibration bursts, like the hammer impacts that generate them, are necessarily spaced apart in time. So time-delayed characteristic backscatter vibration energy from stimulated renal calculi may be sensed by fluid interface vibration detectors in time periods between bursts of transmitted vibration. Thus, both transmitted and characteristic backscatter vibration energy can be detected and distinguished at the same fluid interface because they are, in general, present at different times.

Further, the time-delay associated with characteristic backscatter vibration may be interpreted (e.g., using frag diagnostics) to indicate the stimulation depth or total distance traveled by the transmitted vibration energy and the backscatter vibration energy. And changes in the backscatter vibration's amplitude and/or power spectral density may also (again using frag diagnostics) be used to characterize the composition and/or size of target renal calculi. Thus, information detected by one or more vibration detectors at a fluid interface, as well as estimates of related parameters that can be extracted therefrom, may be particularly helpful when choosing among available treatment options for a particular patient.

The importance of vibration-related information is reflected in the schematic illustrations herein of adaptive lithotripsy systems (see FIGS. 4 and 5). Each illustration includes a block-diagram of at least one adaptive stimulator, each diagram schematically separating the functions of generating and transmitting broad-spectrum vibration for stimulation from the functions of detecting both transmitted vibration energy and band-limited (and time-shifted) backscatter vibration energy. The separated functions schematically emphasize, for example, that changes in backscatter vibration's frequency band limits are reflected as shifts in the vibration energy's PSD. That is, an up-shift in PSD will mean that relatively lower frequencies represent a smaller fraction of the backscatter's total vibration energy. And relatively higher frequencies will be seen to represent a greater portion of the backscatter's total vibration energy. Such an up-shift would occur naturally as stimulation of renal calculi progresses, with backscatter vibration arising in ever-smaller stimulated particles having relatively higher resonant frequencies.

Since backscatter vibration emanates from particles experiencing vibration resonance excitation (i.e., stimulation), changes in the backscatter vibration's PSD can reveal specific changes in the particles' resonance frequencies. And since particles' resonance frequencies are functions of, among other things, particle size and composition (e.g., hardness), analysis of PSD data can directly indicate the local effects of stimulation. In other words, frag diagnostics applied during the stimulation process can provide near-real time information on the changing nature of the stimulated renal calculi. ***Specifically, the extent, speed and range of stimulation generated fragmentation can be estimated through analysis of sequential PSD shifts in band-limited backscatter vibration energy.***

Note that the influence of absolute power levels on backscatter vibration calculations may be significantly reduced through scaling of power measurements (including PSD) to local maxima.

Note also that periodic estimates of the degree of shift in PSD may be used to estimate progress (in near-real time) toward a desired end point for stimulation. Thus, stimulation may be optimized via control of transmitted vibration energy to achieve a predetermined degree of fragmentation. If further fragmentation is desired, one may up-shift the PSD of the originally-transmitted vibration to make more relatively high-frequency stimulation energy available.

Responsiveness of adaptive lithotripsy results to programmed alterations in PSD and/or total transmitted vibration power may depend in part on the electromagnetic coupling (or responsiveness) of a hammer to the electromagnetic hammer driver of an adaptive stimulator and/or to current in the transverse peripheral coil. Adaptive coupling may be achieved via, e.g., a field emission structure comprising an electromagnetic controller having programmable magnetic field polarity reversal and variable magnetic field strength, as seen, e.g., in linear reversible motors. Control of magnetic field strength is optionally via open-loop and/or closed-loop networks associated with the electromagnetic controller. Note that such magnetic field strength control allows the electromagnetic hammer driver to influence hammer movement before, during and after each impact via attractive or repelling forces. See. e.g., the '252 patent for further discussion of such forces.

Note that cyclical changes in magnetic field strength may be characterized by a polarity reversal frequency responsive to vibration electrical signals and/or to a feedback control electrical signal from a programmable stimulator controller. Longitudinal movement of the hammer is thus responsive in part (e.g., via electromagnetic attraction and repulsion) to the electromagnetic hammer driver's cyclical magnetic polarity reversal. For example, longitudinal movement of the hammer striking, flexing, and subsequently rebounding from, the fluid interface may be in-phase with the polarity reversal frequency to generate vibration transmitted by the fluid interface.

Thus, for example, each hammer strike is at least in part a function of magnetic field polarity and strength, and it is followed by a rebound which is at least in part a function of flexure due to elastic properties (e.g., effective elastic modulus) of the hammer and fluid interface. The rebound may also be a function of the electromagnetic hammer driver's magnetic field polarity and strength, as well as the longitudinal magnetic field. The duration of the hammer's entire strike-flexure-rebound cycle is thus controllable; it is termed herein “reflex cycle time” and is measured in seconds. The inverse of reflex cycle time has the same dimensions as frequency (e.g., cycles per second) and is termed “reflex characteristic frequency” herein.

Each hammer strike & rebound applies a mechanical shock to the fluid interface which generates a (relatively-broad) spectrum of stimulation vibration frequencies that are transmitted hydraulically via the fluid interface (and the surrounding fluid) to the patient. The breadth of the generated stimulation vibration spectrum is a reflection of a mechanical shock's duration (i.e., the reflex cycle time). Shortening the reflex cycle time broadens the generated-vibration spectrum (i.e., the spectrum extends to include relatively higher frequencies). The PSD is therefore up-shifted, meaning that more of the total power of the transmitted spectrum is represented in relatively higher frequencies. In this manner, additional stimulation energy (i.e., calculi-fracturing energy) may be directed to relatively smaller fragments because these fragments have resonances at the relatively-higher stimulation vibration frequencies. Thus, an adaptive stimulator's transmitted stimulation vibration energy may be controlled so as to encourage continued calculi fracturing to a predetermined fragment size.

Summarizing the above, hammer rebound movement may be either augmented or impeded by the longitudinal magnetic field and/or the electromagnetic hammer driver's magnetic field polarity and strength, thereby changing reflex cycle time and thus shifting the PSD of stimulation vibration burst spectra generated. That is, either or both of the longitudinal magnetic field and the electromagnetic hammer driver's field emission structure (comprising an electromagnetic controller) can effectively, and in near-real time, tune each stimulation vibration burst spectrum transmitted by the fluid interface for application to (actual or suspected) renal calculi. Such PSD shifting may comprise, for example, altering a transmitted vibration spectrum's bandwidth and/or changing the relative magnitudes of the vibration spectrum's frequency components. In other words, stimulation energy in the form of vibration spectra transmitted via an adaptive stimulator's fluid interface may be subject (in near-real time) to alterations responsive to ongoing results of frag diagnostic calculations. The calculations, in turn, operate on characteristic backscatter vibration data (in the form of vibration electrical signals) to generate feedback control electrical signals.

Note that alternative embodiments of a tunable vibration generator may be described as having the form of a linear electrical motor, the hammer acting as an armature. One such form is seen in railguns, with the armature providing the conducting connection between (parallel) rails. In this (hypothetical) case, opposing currents in the rails (and thus the hammer movement) would be controlled by the electromagnetic hammer driver to achieve the desired characteristic reflex frequency. See, e.g., U.S. Pat. Nos. 8,371,205 and 8,677,877, both incorporated by reference.

Progressive alterations in the character of adaptive stimulation vibration energy applied to actual or suspected renal calculi may include, for example, progressive changes in vibration frequencies present, progressive changes in relative energy levels of vibration frequency components, and/or progressive changes in the total power of a burst of stimulation vibration comprising a plurality of transmitted frequencies. Such changes may be desirable while adaptive stimulation proceeds through a continuum of fracturing (or diagnostic testing for) renal calculi. Success in diagnostic testing for renal calculi, as well as progress in adaptive stimulation of known calculi, is reflected in characteristic backscatter vibration, the character of which changes with continued fracturing and subsequent fragmentation of the calculi. That is, the calculi's absorption of stimulation vibration energy, and near-real-time radiation of backscatter vibration, changes with time. Such changes in backscatter vibration may then be detected (e.g., by a vibration detector) at the adaptive stimulator's fluid interface. The resulting signal may then be fed back to the electromagnetic hammer driver (see, e.g., FIGS. 1-3) and/or transmitted to a programmable controller (see, e.g., FIGS. 4 and 5) for further processing via frag diagnostics.

In an adaptive stimulation array, the electromagnetic hammer driver polarity reversal frequency, instant of hammer strike, and/or characteristic reflex cycle times of each stimulator may be made a function of, e.g., a band-limited portion of backscatter vibration. Constructive or destructive interference of stimulation vibration emanating from stimulators in such an array may occur throughout the range of stimulation vibration, assuring a changing emphasis on vibration at any given frequency and/or within any given frequency band. This minimizes the likelihood of missing critical vibration frequencies needed for diagnosis and/or treatment.

Note that each electromagnetic hammer driver comprises an electromagnetic controller enabling cyclical magnetic polarity reversal characterized by a variable polarity reversal frequency. Note further that longitudinal movement of each hammer is responsive to such cyclical magnetic polarity reversal, and that longitudinal movement of each hammer striking, flexing, and rebounding from, a fluid interface is electively in-phase with a polarity reversal frequency. Additionally note that each transmitted vibration PSD is responsive to adjustable reflex cycle time, and adjustable reflex cycle time may be responsive to at least one timed stimulator shift signal.

Adaptive lithotripsy system embodiments may thus incorporate timed stimulator shift signals responsive to a plurality of vibration electrical signals which are accelerometer generated and/or magnetostrictive-vibration-detector generated. Further, adjustments of PSD via changes in reflex cycle time and fluid interface resonant frequency may be in-phase. So an adaptive lithotripsy array's transmitted PSD may be tunable via shift of one or more adjustable PSD's of transmitted vibration power within a vibration burst. Decreasing at least one adjustable reflex cycle time, or increasing at least one fluid interface resonant frequency, causes up-shift of at least one adjustable PSD to shift relative transmitted vibration power within at least one transmitted vibration burst to relatively higher frequencies for tuning the stimulation array.

Another determinant of transmitted stimulation vibration PSD is the elastic modulus of the hammer's striking face, which may be relatively high (approximately that of mild steel, for example) if a relatively broad spectrum of stimulation vibration is desired. Conversely, a lower hammer striking face modulus of elasticity may be chosen to reduce the highest frequency components of stimulation vibration spectra.

In a first example embodiment, an adaptive stimulator comprises a hollow cylindrical housing having a longitudinal axis, a first end, and a second end, the first end being closed by a fluid interface for transmitting and receiving vibration. The fluid interface comprises at least one vibration detector for producing vibration electrical signals representing vibration transmitted and received by the fluid interface. The fluid interface may comprise a plurality of vibration detectors, each vibration detector having a resonant frequency, and all the resonant frequencies may be similar.

A transverse coil peripheral to and surrounding the fluid interface generates a time-varying longitudinal magnetic field intersecting the fluid interface. The fluid interface may comprise at least one (or a plurality of) disc-shaped thin members, each disc-shaped thin member having a resonant frequency and being oriented substantially perpendicular to the longitudinal magnetic field. One or more disc-shaped thin members may produce vibration electrical signals representing vibration transmitted and received by the fluid interface. At least one said disc-shaped thin member may comprise amorphous ferromagnetic alloy, and the amorphous ferromagnetic alloy may comprise Metglas 2605SC. And at least one disc-shaped thin member's resonant frequency may be responsive to the longitudinal magnetic field. In that case, the fluid interface itself is magnetostrictively responsive to the longitudinal magnetic field because one or more of its disc-shaped thin members has an effective elastic modulus (and thus a related resonant frequency) which is itself magnetostrictively responsive to the longitudinal magnetic field.

An electromagnetic hammer driver reversibly seals the second end, and a hammer is longitudinally movable within the housing between the electromagnetic hammer driver and the fluid interface.

The electromagnetic hammer driver comprises an electromagnetic controller having cyclical magnetic polarity reversal (and thus variable field strength) implemented via, for example, a passive timing network or an embedded microprocessor's stored program. Cyclical magnetic polarity reversal is characterized by a variable polarity reversal frequency. And the fluid interface is magnetostrictively responsive to the longitudinal magnetic field by altering its effective elastic modulus. Longitudinal movement of the hammer striking, flexing, and rebounding from, the fluid interface is responsive (e.g., via electromagnetic attraction and repulsion) to (1) the longitudinal magnetic field and (2) the electromagnetic hammer driver's cyclical magnetic polarity reversal (analogous in part to a linear electrical motor). Further, longitudinal movement of the hammer striking, flexing, and subsequently rebounding from, the fluid interface during a reflex cycle time may be in phase with the polarity reversal frequency to generate vibration transmitted by the fluid interface. Thus, the longitudinal magnetic field is operative on the hammer (to alter reflex cycle time), and on each of the magnetostrictive disc-shaped thin members of the fluid interface (to alter their effective elastic modulus and thus alter their resonant frequencies).

Note that hammer rebound movement may be augmented or impeded by the electromagnetic hammer driver's magnetic field polarity and/or the longitudinal magnetic field, thereby changing reflex cycle time and thus changing the character of vibration spectra generated. In other words, the electromagnetic hammer driver's electromagnetic controller and/or longitudinal magnetic field can effectively, and in near-real time, tune each vibration spectrum transmitted by the fluid interface for application to renal calculi. Such tuning may comprise, e.g., altering a transmitted vibration spectrum's bandwidth and/or changing the relative magnitudes of the vibration spectrum's frequency components. In other words, stimulation energy in the form of vibration spectra transmitted by an adaptive stimulator's fluid interface may be subject (in near-real time) to predetermined alterations.

Such alterations in the character of stimulation energy applied to renal calculi (in the form of relatively broad-band vibration) may include, e.g., changes in vibration frequencies present and/or in relative energy levels of vibration frequency components. Such changes may be desirable while stimulation progresses through a continuum of fracturing of the renal calculi. As progress of stimulation is reflected in progressive fracturing and/or fragmentation of the renal calculi, the calculi's absorption of stimulation energy changes in a time-varying manner. Changes in absorbed energy, in turn, cause changes in backscattered vibration that may be sensed by an accelerometer and/or vibration detector at the fluid interface. The resulting electrical signal may then be fed back to the electromagnetic hammer driver (e.g., by cable or wirelessly) as described herein.

The invention thus facilitates a form of closed-loop (feedback) control of the stimulation process that may be optimized (i.e., yielding better results from less stimulation). One might choose, for example, to emphasize relatively lower frequency stimulation energy initially, followed by adaptively increasing relatively higher frequency vibration spectrum components as stimulation progresses. Individual adaptive stimulators of the invention can support such an optimization strategy inherently because they naturally produce relatively broad vibration spectra having controllable amplitude and PSD (rather than single-frequency vibration).

Should a greater frequency range be desired than that obtainable from a single adaptive stimulator, a plurality of such stimulators may be interconnected in an adaptive stimulator array. Operation of such an array may be controlled via, for example, a programmable stimulator controller comprising a reflex cycle time estimator and a fluid interface resonant frequency estimator. The programmable stimulator controller may govern each electromagnetic hammer driver through its electromagnetic controller having cyclical magnetic polarity reversal characterized by a variable polarity reversal frequency. Longitudinal movement of each hammer is thus responsive to electromagnetic hammer driver cyclical magnetic polarity reversal for striking, flexing, and rebounding from, the fluid interface during a reflex cycle time. The inverse of each reflex cycle time is a reflex characteristic frequency, and each time-varying longitudinal magnetic field may be in phase with one reflex characteristic frequency.

Second and third example embodiments of adaptive stimulators are analogous in several respects to the first example embodiment, with the fluid interface comprising at least one vibration detector for producing vibration electrical signals representing vibration of the fluid interface due to both transmitted and backscattered vibration. As in the first example, the electromagnetic hammer driver comprises an electromagnetic controller having cyclical magnetic polarity reversal (and thus variable field strength).

Cyclical magnetic polarity reversal is characterized by a polarity reversal frequency which is variable. The electromagnetic hammer driver controller receives the vibration electrical signal (via, e.g., an electrical cable or wirelessly) and processes (e.g., via a microprocessor executing a stored program) the signal to produce excitation for the electromagnetic hammer driver electromagnet for control of its cyclical magnetic polarity reversal (and thus its polarity reversal frequency). The polarity reversal frequency is thus responsive to the vibration electrical signals. And since the hammer is responsive to the electromagnetic hammer driver and the longitudinal magnetic field, longitudinal movement of the hammer striking, flexing, and rebounding from, the fluid interface may be in phase with the time-varying longitudinal magnetic field and/or with the polarity reversal frequency during predetermined portions of stimulation.

Further, longitudinal hammer movement, as noted above, is associated with a reflex characteristic frequency. In certain embodiments, the reflex characteristic frequency may approximate or equal the polarity reversal frequency.

Note that part of the vibration sensed at the fluid interface includes characteristic backscattered vibration that may contain information on the progress of renal calculi stimulation (e.g., the degree of calculi fracturing and/or fragmentation, including the size and/or composition of calculi fragments) induced in part by vibration earlier transmitted from the fluid interface. (See U.S. Pat. No. 8,535,250, incorporated by reference).

Note also that the electromagnetic hammer driver's polarity and field strength may also or alternatively be responsive (e.g., via integrated control electronics and windings of the electromagnet) to vibration electrical signals from one or more of the fluid interface's vibration detectors (the electrical signals being, in-part, functions of the amplitude and frequency of backscattered vibration received by the fluid interface). The electromagnetic hammer driver's polarity and field strength, in turn, influence hammer position and velocity determined by, e.g., laser (or other electromagnetic) ranging of hammer position and hammer velocity relative to the hammer driver. Reception of the backscattered vibration, in either case, allows near-real-time estimation of the degree of stimulation imposed by the adaptive stimulator.

PSD shifts that are part of adaptive lithotripsy allow continuous (i.e., swept-frequency) tailoring of total impulse-generated resonant frequency power. In diagnostic applications, this tailoring is accomplished in nearly real time to minimize total transmitted vibration power while still stimulating resonance vibration and characteristic backscatter vibration from the calculi whose existence and location are being sought. Because of the relatively brief periods of characteristic backscatter vibration that may be detected between frequency sweeps of transmitted vibration, automatic warning of renal calculi may be desired for the lithotripter operator via the outputs of a series of narrow-band filters within a programmable stimulator controller for vibration electrical signals.

The filters may conveniently be located within the fluid interface resonant frequency estimator portion of the controller. Filter outputs may, for example be coupled to a two-dimensional visual display (e.g., LED lights) with amplitude along the vertical axis and frequency band along the horizontal axis. Such a display would graphically indicate PSD up-shifts as concentrations of light moving toward the upper right hand corner, while PSD down-shifts would be seen as concentrations of light moving toward the upper left hand corner.

### BRIEF DESCRIPTION OF THE DRAWINGS

FIG. 1 illustrates a schematic 3-dimensional view of an adaptive stimulator comprising a vibration detector and a tunable vibration generator. A hammer is longitudinally movable within a hollow cylindrical housing, one end of the housing being closed by a fluid interface, and the other end being closed by an electromagnetic hammer driver. The fluid interface is shown with a MEMS accelerometer for detecting vibration of the interface.

FIG. 2 illustrates a schematic 3-dimensional exploded view of the adaptive stimulator embodiment of FIG. 1, a first electrical cable being shown to schematically indicate a feedback path (for an accelerometer signal) from the accelerometer to the electromagnetic hammer driver. A second electrical cable is shown to schematically indicate an interconnection path for, e.g., communication with one or more additional stimulators and/or associated equipment such as a programmable controller.

FIG. 3 illustrates a schematic 3-dimensional exploded view of an adaptive stimulator embodiment that differs from the embodiment of FIGS. 1 and 2 in part because it comprises a fluid interface comprising three disc-shaped thin members. Electrical leads signify that each disc-shaped thin member functions as a vibration detector, and electrical leads also draw attention to an electromagnetic hammer driver and a transverse peripheral coil for creating a longitudinal magnetic field.

FIG. 4 schematically illustrates a 2-dimensional view of major components, subsystems, and interconnections of an adaptive lithotripsy system comprising the adaptive stimulator embodiment of FIG. 3. As aids to orientation, communication pathways are indicated between stimulator components (tunable vibration generator and vibration detector), a stimulator controller running frag diagnostics, and estimators for reflex cycle time and fluid interface resonant frequency. Schematic pathways are shown for transmitted stimulation vibration energy and for backscatter vibration energy.

FIG. 5 schematically illustrates an embodiment of an adaptive lithotripsy system analogous in part to that in FIG. 4, but differing in the presence of a linear array of 3 adaptive stimulators instead of the single adaptive stimulator of FIG. 4. Appropriate timing of stimulation vibration bursts from each stimulator facilitates directional propagation of combined-vibration wave fronts. Further, feedback-control of total transmitted power and transmitted vibration PSD make the embodiment exceptionally flexible for diagnosis and treatment.

### DETAILED DESCRIPTION

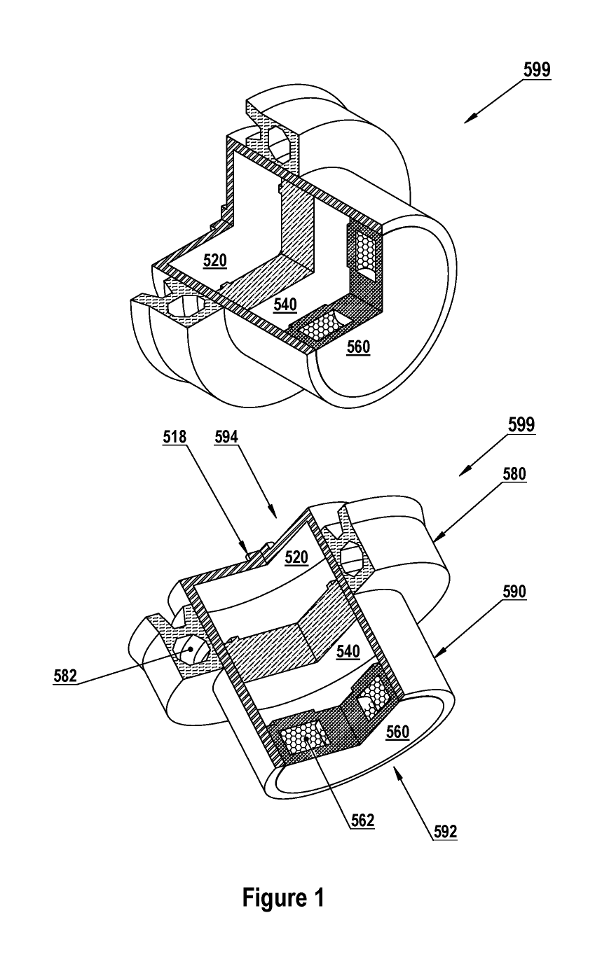

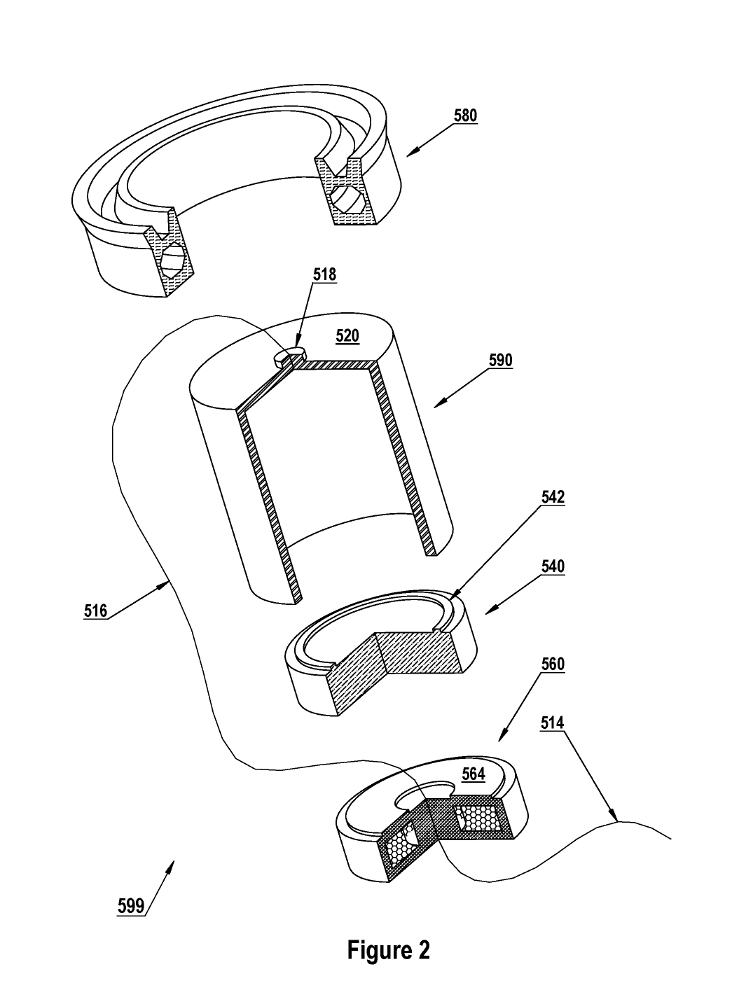

FIGS. 1 and 2 illustrate partial schematic 3-dimensional views of an adaptive stimulator of class 599, FIG. 2 being an exploded view. Numerical labels may appear in only one view. A hollow cylindrical housing 590 has a longitudinal axis, a first end 594, and a second end 592. First end 594 is closed by fluid interface 520 for transmitting and receiving vibration. Fluid interface 520 comprises at least one accelerometer 518 for producing a vibration electrical signal (i.e., an accelerometer-generated feedback signal) representing vibration transmitted and received via fluid interface 520.

Electromagnetic hammer driver 560 (comprising a field emission structure which itself comprises electromagnet face 564 and electromagnetic controller 562) reversibly seals second end 592, and hammer (or movable mass) 540 is longitudinally movable within cylindrical housing 590 between electromagnetic hammer driver 560 and fluid interface 520. In some embodiments, hammer 540 may itself be a field emission structure consisting of a permanent magnet (or a plurality of permanent magnets). Polarity of any such permanent magnets is not specified because it would be assigned in light of the electromagnetic controller 562. Alternatively, hammer 540 may be analogous in part to the armature of a linear electric motor, as in a railgun. (See, e.g., the '205 and '877 patents noted above). Note that the above accelerometer-generated vibration electrical signal may be augmented by a sensorless control means (e.g., controlling operating parameters of electromagnetic controller 562 such as magnetic field strength and polarity) in free piston embodiments of the adaptive stimulator. (See, e.g., U.S. Pat. No. 6,883,333, incorporated by reference).

Thus, hammer 540 is responsive to the magnetic field emitted by electromagnet face 564 of electromagnetic hammer driver 560 for striking, flexing, and rebounding from, fluid interface 520. The duration of each such striking, flexure and rebounding cycle (termed herein the “reflex cycle time”) has the dimension of seconds. And the inverse of this duration has the dimension of frequency. Hence, the term herein “characteristic reflex frequency” is the inverse of a reflex cycle time, and the reflex cycle time itself is inversely proportional to the bandwidth of transmitted vibration spectra resulting from each hammer strike and rebound from fluid interface 520.

Fluid interface 520 transmits vibration spectra generated by hammer impacts on fluid interface 520 as well as receiving backscatter vibration from renal calculi excited by a stimulator of class 599. Fluid interface 520 comprises, for example, a MEMS accelerometer 518 for producing an accelerometer signal representing vibration transmitted and received by fluid interface 520. (See MicroElectro-Mechanical Systems in Harris, pp. 10-26, 10-27).

Hammer 540 comprises a striking face 542 (see FIG. 2) which has a predetermined modulus of elasticity (e.g., that of mild steel, about 29,000,000 psi) which can interact with the effective elastic modulus of fluid interface 520. In an illustrative example, interaction of the two suggested moduli of elasticity predetermines a relatively short reflex cycle time for hammer 540, which is associated with a corresponding relatively broad-spectrum of vibration to be transmitted by fluid interface 520. In other words, striking face 542 strikes fluid interface 520 and rebounds to produce a relatively short-duration, high-amplitude mechanical shock. (See, e.g., Harris p. 10.31).

Both FIGS. 1 and 2 schematically illustrate a tunable resilient circumferential seal 580 for sealing cylindrical housing 590 within a lithotripsy bath, thus partially isolating vibration transmitted by fluid interface 520 within the bath. Circumferential seal 580 comprises at least one circular tubular area 582 which may contain at least one shear-thickening fluid which may be useful in part for tuning purposes. The shear-thickening fluid, in turn, may comprise nanoparticles for, e.g., facilitating heat scavenging.

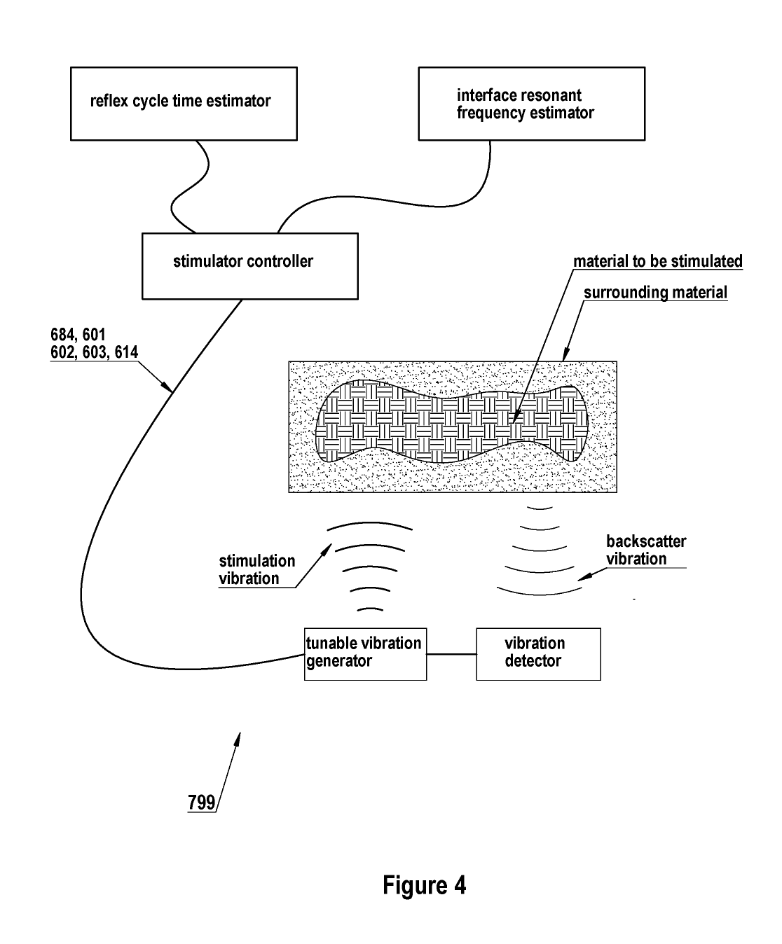

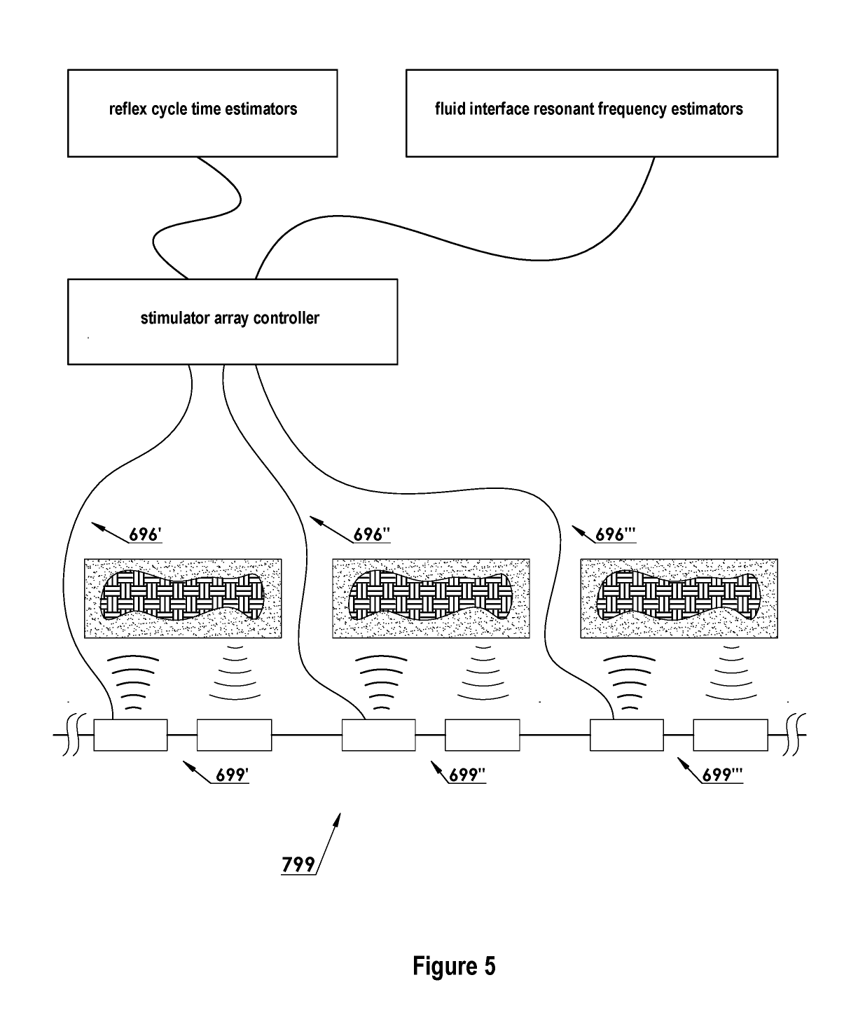

FIG. 2 also schematically illustrates a first electrical power/data cable 516 for carrying vibration electrical signals (representing vibration data transmitted by and/or received by fluid interface 520) from accelerometer 518 to electromagnetic hammer driver 560. A second electrical power/data cable 514 also connects to electromagnetic hammer driver 560 of each adaptive stimulator to schematically represent interconnection of two or more such stimulators (to form an adaptive stimulator array) and/or for connecting one or more adaptive stimulators to related equipment (e.g., a programmable stimulator controller as shown in FIGS. 4 and 5). Vibration electrical signals provide feedback on transmitted vibration and also on received characteristic backscatter vibration to electromagnetic hammer driver 560.

While accelerometer-mediated feedback may be desired for tailoring stimulation to specific renal calculi and/or to progress in producing desired vibration frequencies for diagnosis and/or fracture of calculi, predetermined stimulation protocols may be used instead to simplify operations and/or lower costs.

Note that transmitted vibration power levels suitable for diagnosis may be significantly lower than vibration power levels needed for fracturing calculi. Since lower vibration power levels are more consistent with patient comfort and safety, screening diagnostic tests will typically employ adaptive lithotripsy systems adjusted for minimum transmitted vibration power levels needed to generate detectable backscatter vibration from any renal calculi that may be present.

In certain embodiments, frag diagnostic software and data to implement sensorless control via operating parameters (e.g., magnetic field strength and polarity) of electromagnetic controller 562, or to implement feedback control incorporating accelerometer 518, are conveniently stored and executed in a microprocessor (located, e.g., in electromagnetic controller 562). (See, e.g., U.S. Pat. No. 8,386,040, incorporated by reference). See FIGS. 5 and 6 of the '040 patent, for example, with their accompanying specification.

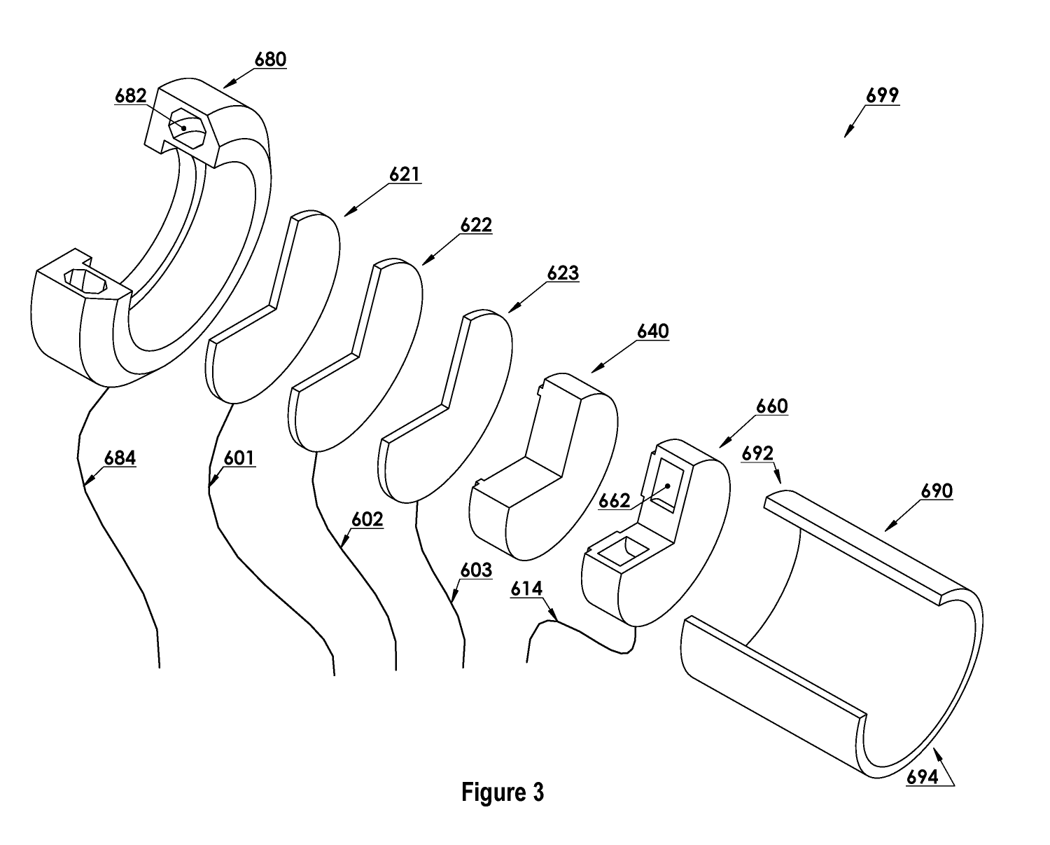

Note, however, that while certain of the electrodynamic control characteristics of an adaptive stimulator may be represented in earlier devices, the adaptive stimulator's reliance on mechanical shock (e.g., generated by hammer strike and rebound) to generate tuned vibration (i.e., vibration characterized by approximately predetermined magnitude and/or frequency and/or PSD) imposes unique requirements indicated by the dynamic responsiveness of certain mechanical structures (e.g., hammers and fluid interfaces) to electromagnetic effects of field-emitting components (e.g., electromagnets and electret materials) as described herein. Variability of stimulation vibration is further responsive to one or more programmable stimulator controllers via, e.g., the power/data cable 514, and/or an analogous-in-part combined electrical cable (see, e.g., FIG. 3). Such responsiveness may extend to other adaptive stimulators and/or to other auxiliary equipment (see, e.g., FIG. 5).

Note also that in addition to individual applications of an adaptive stimulator, two or more such stimulators may operate in a combined adaptive stimulator array during a given stage of adaptive lithotripsy. A single adaptive stimulator or an interconnected adaptive stimulator array may be programmed in near-real time to alter stimulation parameters in response to changing conditions in biologic materials to be adaptively stimulated. A record of such changes, together with results, guides future changes to increase stimulation efficiency.

In summary, the responsiveness of certain components of an adaptive stimulator to other components and/or to parameter relationships facilitates operational advantages in various alternative stimulator embodiments. Examples involving such responsiveness and/or parameter relationships include, but are not limited to: (1) electromagnetic hammer driver 560 comprises a field emission structure comprising an electromagnetic controller 562 having cyclical magnetic polarity reversal characterized by a variable polarity reversal frequency; (2) longitudinal movement of hammer 540 (or movable mass) striking, flexing, and rebounding from, the fluid interface 520 is responsive to the electromagnetic hammer driver cyclical magnetic polarity reversal; (3) longitudinal movement of hammer 540 striking, flexing, and rebounding from, fluid interface 520 may be in-phase with the polarity reversal frequency to generate vibration transmitted by fluid interface 520; (4) the polarity reversal frequency of electromagnetic hammer driver 560 may be responsive to accelerometer 518's vibration electrical signal, and thus responsive to vibration sensed by accelerometer 518; (5) longitudinal movement of hammer 540 may be in-phase with the polarity reversal frequency; (6) longitudinal movement of hammer 540 striking, flexing, and rebounding from, fluid interface 520 has a characteristic reflex frequency which is the inverse of the reflex cycle time; (7) the hammer 540 characteristic reflex frequency may be in-phase with polarity reversal and; (8) the reflex cycle time is a function of the cyclical magnetic polarity of electromagnetic hammer driver 560 and/or the moduli of elasticity of striking face 542 of hammer 540 and that of fluid interface 520.

FIG. 3 illustrates a schematic 3-dimensional exploded view of one embodiment of an adaptive stimulator of class 699. Stimulators of class 699 share several structural and functional features analogous to structural and functional features of an adaptive stimulator of class 599 (schematically illustrated in FIGS. 1 and 2). But stimulators of class 699 differ materially in several respects from the stimulator illustrated in FIGS. 1 and 2. Subsequent description herein identified with class 699 or FIG. 3 should be understood as relating to a group comprising stimulators which demonstrate common structural features of, as well as one or more material structural and/or functional differences from, stimulators of class 599.

Within class 699, material differences among embodiments include, but are not limited to (1) the number of disc-shaped thin members comprising a fluid interface, (2) the composition of individual disc-shaped thin members (e.g., various magnetostrictively-responsive amorphous ferromagnetic alloys), (3) surface shapes of disc-shaped thin members, (4) manufacturing treatment of magnetostrictively-responsive disc-shaped thin members such as annealing in a magnetic field which alters magnetostrictive responsiveness, (5) vibration damping characteristics, (6) methods of assembling a plurality of disc-shaped thin members, such as lamination or mechanical compression, to form a fluid interface, and (7) electrical interconnection of one or more disc-shaped thin members with other stimulator components and/or other components of adaptive lithotripsy systems (see, e.g., FIGS. 4 and 5).

Hence, FIG. 3 schematically illustrates certain construction features of an example adaptive stimulator of class 699 which are not limited to a specific embodiment. The example comprises a hollow cylindrical housing 690 having a longitudinal axis, a first end 692, and a second end 694. First end 692 is closed by a fluid interface for transmitting and receiving vibration. In general, fluid interfaces of stimulators of class 699 each comprise one or more disc-shaped thin members which are analogous-in-part to disc-shaped thin member 621, disc-shaped thin member 622 and/or disc-shaped thin member 623. The illustrated fluid interface embodiment 621/622/623 comprises the three illustrated disc-shaped thin members in a compact (e.g., laminated) subassembly for purposes of description only, but alternate fluid interface embodiments of the invention may contain more or fewer disc-shaped thin members. At least one disc-shaped thin member within fluid interface 621/622/623 comprises ferromagnetic amorphous alloy, the effective elastic modulus of which is magnetostrictively-responsive to a time-varying longitudinal magnetic field created by electrical current in peripheral coil 682 which is schematically shown as enclosed in coil form 680. The longitudinal magnetic field influences the effective hardness of, and thus the resonant frequencies of: (1) at least one disc-shaped thin member 621, 622 and/or 623 and (2) the fluid interface 621/622/623 as a whole. Further, at least one disc-shaped thin member within fluid interface 621/622/623 comprises a vibration detector for generating a vibration electrical signals representing both vibration transmitted and characteristic backscatter vibration received via fluid interface 621/622/623.

Continuing with a description of FIG. 3, second end 694 of hollow cylindrical housing 690 is closed by electromagnetic hammer driver 660 (comprising a field emission structure which itself comprises electromagnetic controller 662 within electromagnetic hammer driver 660). Hammer (or movable mass) 640 is longitudinally movable within housing 690 between electromagnetic hammer driver 660 and fluid interface 621/622/623. In some embodiments, hammer 640 may itself be a field emission structure consisting of a permanent magnet (or consisting of a plurality of permanent magnets). Polarity of any such permanent magnets is not specified because it would be assigned in light of field emission from the electromagnetic controller 662. Alternatively, hammer 640 may be analogous in part to the armature of a linear electric motor, as in a railgun. (See, e.g., the '205 and '877 patents noted above).

Note that the longitudinal magnetic field is operative on hammer 640 (to alter reflex cycle time), and on each of the magnetostrictive disc-shaped thin members of fluid interface 621/622/623 (to alter their effective elastic modulus and thus alter their resonant frequencies).

Note also that the above vibration electrical signals representing vibration transmitted and/or received via fluid interface 621/622/623 may be augmented by sensorless control means (e.g., controlling operating parameters of electromagnetic controller 662 such as magnetic field strength and polarity) in free piston embodiments of adaptive stimulators of class 699.

FIG. 4 schematically illustrates a 2-dimensional view of major components and interconnections of adaptive lithotripsy system 798, together with brief explanatory labels and comments on component functions. As aids to orientation, a schematic lithotripsy target (i.e. material to be stimulated) is shown. Stimulation vibration and backscatter vibration (hydraulic) pathways are schematically illustrated for transmitting broad-spectrum vibration to, and receiving band-limited backscatter vibration from, material to be stimulated (e.g., renal calculi).

Adaptive lithotripsy system 798 schematically illustrated in FIG. 4 is relatively sophisticated, employing several structures, functions and interactions that appear in different invention embodiments. For example, closed-loop feedback-control of fluid interface resonant frequency is graphically indicated as a function of the stimulator controller. Analogously, closed-loop feedback-control of reflex cycle time is also graphically indicated as a function of the stimulator controller. Adjustment of either (1) fluid interface resonant frequency or (2) reflex cycle time (or both) may be implemented via the stimulator controller to up-shift or down-shift transmitted vibration PSD. Shifting PSD effectively narrows the relatively broad spectrum of transmitted vibration by causing vibration power to be relatively concentrated in predetermined (effectively narrowed) portions of the transmitted frequency spectrum.

Note that FIG. 4 necessarily represents an application of adaptive stimulators of class 699 because such stimulators feature PSD shifting by adjustments of reflex cycle time and/or fluid interface resonant frequency. In contrast, PSD shifting in an adaptive lithotripsy system featuring adaptive stimulators of class 599 is a function of reflex cycle time.

Note further that the labeling of an adaptive stimulator of class 699 in FIG. 4, comprising a tunable vibration generator combined with a vibration detector, emphasizes that the vibration detector is co-located with the vibration generator. One embodiment demonstrating such co-location of tunable vibration generator and vibration detector is that shown in FIG. 3, where one or more disc-shaped thin members function as vibration detectors while the fluid interface as a whole transmits stimulating vibration (and receives backscatter vibration). An alternative embodiment featuring co-location of vibration generator and vibration detector is that of the adaptive stimulator of class 599 in FIGS. 1 and 2, where an accelerometer (i.e., a vibration detector) is mounted directly on a fluid interface which transmits and receives vibration.

Another difference between adaptive stimulators of class 599 and those of class 699 is that in stimulators of the latter class, the fluid interface resonant frequency estimator and the reflex cycle time estimator, while functions of the (programmable) stimulator controller, rely on data from peripheral coil 682 (regarding longitudinal magnetic field strength). At least one disc-shaped thin member of the adaptive stimulator of FIG. 3 is subject to magnetostrictive effects on the effective elastic modulus of the thin member. So the longitudinal magnetic field is operative on (1) the hammer (to alter reflex cycle time), and on (2) each of the magnetostrictive disc-shaped thin members of the fluid interface (to alter their effective elastic modulus and thus alter their resonant frequencies).

Note that data from one or more of the disc-shaped thin members (electrical cables 601, 602 and/or 603), peripheral coil 682 (electrical cable 684) and electromagnetic controller 662 (electrical cable 614) are transmitted to the stimulator controller via combined electrical cable 696.

Notwithstanding the above differences, in adaptive stimulators of both class 599 and class 699, a field emission structure may be responsive to at least one control signal (e.g., timed stimulator transmission signals and/or stimulator shift signals). Such responsiveness to at least one control signal is achieved, e.g., by emitting one or more electric and/or magnetic fields which are functions of at least one control signal as sensed by the field emission structure through change in one or more field emission structure electrical parameters. Thus, vibration transmitted by an adaptive stimulator (either class 599 or 699) may have a predetermined PSD which is a function of its reflex cycle time. The reflex cycle time, in turn, is dependent-in-part on one or more field emission structures that are themselves responsive to at least one control signal (e.g., a stimulator shift signal). A stimulator shift signal, in turn, may be responsive to vibration electrical signals via power/data cable 516 in FIG. 2 (class 599) or electrical signals via electrical cable 614 (within combined electrical cable 696) in FIG. 3 (class 699).

FIG. 5 schematically illustrates an embodiment of an adaptive lithotripsy system 799 which differs from adaptive lithotripsy system embodiment 798 shown in FIG. 4. A portion of the 2-dimensional stimulation system view of FIG. 4 is reproduced in FIG. 5, but differences between FIGS. 4 and 5 include replacement of a single adaptive stimulator of class 699 (in FIG. 4) with a linear array comprising three analogous adaptive stimulators (699′, 699″ and 699′″) in FIG. 5. Descriptions of functional features of stimulators in FIG. 5 resemble (in-part) analogous descriptions of the stimulator in FIG. 4, but adaptive lithotripsy system 799 combines impulse-generated swept-frequency stimulation vibration with timed signals to provide adaptive stimulation via a directionally propagated array vibration wave front. Swept-frequency stimulation vibration arises from cyclical up-shifts and down-shifts of the PSD of impulse-generated stimulation vibration. The cyclical PSD shifts, in turn, are achieved via closed-loop feedback-control of the impulse-generated vibration produced by stimulator linear array 699′/699″/699′″. PSD's and fluid interface resonant frequencies of the array stimulators may be individually adjusted for resonance excitation, fracturing and subsequent fragmentation of renal calculi at varying distances from the array.

Stimulation linear array 699′/699″/699′″ may behave in-part in a manner analogous to that of a phased-array antenna. For example, elective discrete time delays among sequential transmission times for vibration bursts from each stimulator in array 699′/699″/699′″ are controlled via timed stimulator transmission signals from the (programmable) stimulator array controller so as to exert control over the propagation direction of the combined stimulation vibration wave front (i.e., control over the directionally propagated array vibration wave front). Timed stimulator transmission signals, in turn, may have a phase relation (e.g., in-phase) with (1) cyclically-varying fluid interface resonant frequencies and/or, (2) cyclically-varying hammer impact reflex cycle times and/or, (3) cyclically varying total transmitted vibration power.

Further, other timing issues affect vibration from each adaptive stimulator in linear array 699′/699″/699′″. For example, differences in individual reflex cycle times among the stimulators affect their individual PSD's. Adjustable reflex cycle times, in turn, may reflect changes in electrical parameters (e.g., current in peripheral coil 682, magnetic field polarity, magnetic field strength, and/or the phase relationship of the time-varying longitudinal magnetic field and/or the stimulator electromagnetic hammer driver polarity reversal to hammer strike). Variability in adjustable reflex cycle times (e.g., non-uniform reflex cycle times) may also be responsive to stimulator shift signals from the (programmable) stimulator array controller. Such variability may result in vibration interference among stimulators in a spatial array. Both constructive interference (i.e., increase in amplitude) at one or more frequencies and destructive interference (i.e., decrease in amplitude) at other frequencies are likely, thus electively providing higher stimulation vibration energy levels at a plurality of discrete frequencies within a vibration burst.

What is claimed is:

1. An adaptive stimulator comprising
a hollow cylindrical housing having a longitudinal axis, a first end, and a second end, said first end being closed by a fluid interface for transmitting and receiving vibration, said fluid interface comprising at least one vibration detector for producing vibration electrical signals representing vibration transmitted and received by said fluid interface;
a transverse coil peripheral to and surrounding said fluid interface, said transverse coil for generating a time-varying longitudinal magnetic field intersecting said fluid interface;
an electromagnetic hammer driver reversibly sealing said second end; and
a hammer longitudinally movable within said housing between said electromagnetic hammer driver and said fluid interface;
wherein said electromagnetic hammer driver comprises an electromagnetic controller having cyclical magnetic polarity reversal characterized by a variable polarity reversal frequency;
wherein said fluid interface is magnetostrictively responsive to said longitudinal magnetic field by altering its effective elastic modulus;
wherein longitudinal movement of said hammer is responsive to said electromagnetic hammer driver cyclical magnetic polarity reversal for striking, flexing, and rebounding from, said fluid interface; and
wherein longitudinal movement of said hammer striking, flexing, and rebounding from, said fluid interface is in phase with said time-varying longitudinal magnetic field.

2. The stimulator of claim 1 wherein said fluid interface comprises a plurality of vibration detectors, each said vibration detector having a resonant frequency.

3. The stimulator of claim 2 wherein all said vibration detector resonant frequencies are similar.

4. The stimulator of claim 1 wherein said fluid interface comprises at least one disc-shaped thin member, each said disc-shaped thin member having a resonant frequency and being oriented substantially perpendicular to said longitudinal magnetic field.

5. The stimulator of claim 4 wherein at least one said disc-shaped thin member comprises amorphous ferromagnetic alloy.

6. The stimulator of claim 5 wherein said amorphous ferromagnetic alloy comprises Metglas 2605SC.

7. The stimulator of claim 4 wherein at least one said disc-shaped thin member's resonant frequency is responsive to said longitudinal magnetic field.

8. The stimulator of claim 4 wherein said fluid interface comprises a plurality of said disc-shaped thin members.

9. The stimulator of claim 8 wherein each said disc-shaped thin member produces said vibration electrical signals representing vibration transmitted and received by said fluid interface.

10. An adaptive stimulator comprising
a hollow cylindrical housing having a longitudinal axis, a first end, and a second end, said first end being closed by a fluid interface for transmitting and receiving vibration, said fluid interface comprising at least one vibration detector for producing vibration electrical signals representing vibration transmitted and received by said fluid interface;
a transverse coil peripheral to and surrounding said fluid interface, said transverse coil for generating a time-varying longitudinal magnetic field intersecting said fluid interface;
an electromagnetic hammer driver reversibly sealing said second end; and
a hammer longitudinally movable within said housing between said electromagnetic hammer driver and said fluid interface, said hammer being responsive to said electromagnetic hammer driver for striking, flexing, and rebounding from, said fluid interface;
wherein said electromagnetic hammer driver comprises an electromagnetic controller having cyclical magnetic polarity reversal characterized by a variable polarity reversal frequency;
wherein said polarity reversal frequency is responsive to said vibration electrical signals; and
wherein longitudinal movement of said hammer is in phase with said polarity reversal frequency.

11. The stimulator of claim 10 wherein said fluid interface comprises a plurality of vibration detectors, each said vibration detector having a resonant frequency.

12. The stimulator of claim 11 wherein all said vibration detector resonant frequencies are similar.

13. The stimulator of claim 10 wherein said fluid interface comprises at least one disc-shaped thin member, each said disc-shaped thin member having a resonant frequency and being oriented substantially perpendicular to said longitudinal magnetic field.

14. The stimulator of claim 13 wherein at least one said disc-shaped thin member comprises amorphous ferromagnetic alloy.

15. The stimulator of claim 14 wherein said amorphous ferromagnetic alloy comprises Metglas 2605SC.

16. An adaptive stimulator array comprising a plurality of adaptive stimulators, all said stimulators being connected to a programmable stimulator controller comprising a reflex cycle time estimator and a fluid interface resonant frequency estimator, and each said stimulator comprising
a hollow cylindrical housing having a longitudinal axis, a first end, and a second end, said first end being closed by a fluid interface for transmitting and receiving vibration, said fluid interface comprising at least one vibration detector for producing vibration electrical signals representing vibration transmitted and received by said fluid interface;
a transverse coil peripheral to and surrounding said fluid interface, said transverse coil for generating a time-varying longitudinal magnetic field intersecting said fluid interface;
an electromagnetic hammer driver reversibly sealing said second end; and
a hammer longitudinally movable within said housing between said electromagnetic hammer driver and said fluid interface;
wherein each said electromagnetic hammer driver comprises an electromagnetic controller having cyclical magnetic polarity reversal characterized by a variable polarity reversal frequency;
wherein longitudinal movement of each said hammer is responsive to said electromagnetic hammer driver cyclical magnetic polarity reversal for striking, flexing, and rebounding from, said fluid interface during a reflex cycle time;
wherein the inverse of each said reflex cycle time is a reflex characteristic frequency; and
wherein each said time-varying longitudinal magnetic field is in phase with one said reflex characteristic frequency.

17. The stimulator array of claim 16 wherein each said fluid interface comprises a plurality of vibration detectors, each said vibration detector having a resonant frequency.

18. The stimulator array of claim 17 wherein all said vibration detector resonant frequencies are similar.

19. The stimulator array of claim 16 wherein each said fluid interface comprises at least one disc-shaped thin member, each said disc-shaped thin member being oriented substantially perpendicular to said longitudinal magnetic field.

20. The stimulator array of claim 19 wherein at least one said disc-shaped thin member comprises amorphous ferromagnetic alloy.
